(19) United States
(12) Patent Application Publication
(10) Pub. No.: US 2026/0248521 A1
(43) Pub. Date: Aug. 27, 2026

(54) SHOCK WAVE CATHETER AND SHOCK WAVE CATHETER SYSTEM

(71) Applicant: Shockwave Medical, Inc., Santa Clara, CA (US)

(72) Inventors: David MOOSAVI, Castro Valley, CA (US); Jason B. FRANCO, Milpitas, CA (US); Jenna M. LIESKE, San Mateo, CA (US); Andrew James HUDON, Tabernash, CO (US); Lawrence John FORMOSA, Campbell, CA (US); Tim Ngoc HUYNH, Milpitas, CA (US); Thi Hong Nhung TRUONG, Lathrop, CA (US); Matthew Tyler SURBER, San Jose, CA (US)

(73) Assignee: Shockwave Medical, Inc., Santa Clara, CA (US)

(21) Appl. No.: 19/643,315

(22) Filed: Apr. 9, 2026

Related U.S. Application Data
(63) Continuation of application No. 19/319,045, filed on Sep. 4, 2025.
(60) Provisional application No. 63/691,815, filed on Sep. 6, 2024.
(60) Provisional application No. 63/783,153, filed on Apr. 3, 2025.

(51) Int. Cl. A61B 17/22; A61B 17/00; A61M 25/00

(52) U.S. Cl. CPC A61B 17/22022; A61B 2017/00243; A61B 2017/00292; A61M 25/0068

(57) ABSTRACT

A catheter for generating shock waves, the catheter comprising: a catheter body; an emitter support member configured to extend at least partially distally of a distal end of the catheter body, where the emitter support member comprises a pre-formed distal portion, where the pre-formed distal portion is configured to coil into an annular loop around at least a portion of a longitudinal axis of the catheter body; a plurality of shock wave emitters mounted to the emitter support member; and an enclosure enclosing the plurality of shock wave emitters.

### CROSS REFERENCE TO RELATED APPLICATIONS

This application is a Continuation of U.S. Non-Provisional application Ser. No. 19/319,045, filed Sep. 4, 2025, which claims priority to U.S. Provisional Application No. 63/691,815, filed Sep. 6, 2024, and U.S. Provisional Application No. 63/783,153, filed Apr. 3, 2025, the entire contents of each of which are incorporated herein by reference in their entirety.

### FIELD OF THE DISCLOSURE

The present disclosure relates generally to the field of medical devices and methods, and more specifically to acoustic energy generating assemblies for inclusion in catheter devices used for treating lesions in a body lumen and tissues, such as calcified lesions and occlusions in the cardiovascular system or on structural heart anatomy.

### BACKGROUND

The accumulation of calcium in a patient's blood vessels, tissues, or other organs can cause calcification that may disrupt organ function and lead to health issues for the patient. For example, when vascular plaque builds up along and in the walls of the coronary arteries, the accumulation can narrow the passageway of the vessel (referred to as stenosis) and restrict blood flow to the heart muscle, which can eventually lead to a heart attack. Treating stenosis is even more challenging when the plaque becomes hardened due to calcification.

A wide variety of catheters have been developed for treating stenotic blood vessels that are narrowed by the progressive growth and accumulation of plaque, a condition also known as atherosclerosis. For example, treatment systems for percutaneous coronary angioplasty or peripheral angioplasty use angioplasty balloons to dilate a calcified lesion and restore normal blood flow in a vessel. In these types of procedures, a catheter carrying a balloon is advanced into the vasculature along a guide wire until the balloon is aligned with a target lesion. The balloon is then pressurized (normally to greater than 10 atm), causing the balloon to expand in a vessel to push plaques back into the vessel wall and dilate occluded regions of vasculature. A particular focus is to treat calcified lesions of plaque in the vasculature associated with arterial disease. When treating calcified lesions, it is important to minimize damage to surrounding soft tissues while still breaking up the lesion as much as possible.

However, traditional dilation balloon angioplasty therapies may not work with calcified tissue because the calcium in the atherosclerotic plaque hardens the lesion, resisting the mechanical force of balloon expansion. The resistance can result in more procedural complication and vessel damage because the high-pressure balloons preferentially expand away from the hard calcified tissue. The predisposition of the ballon to expand in a direction of lower resistance increases the risk of major dissection or perforation of the vessel, often at the ends of a lesion at the interface between healthy tissue and calcified tissue (i.e., where the balloon encounters soft tissue). In the case of an eccentric calcified lesion where the hardened region is biased on a side of a vessel, the expansion ends up going preferentially in the direction opposite of the calcified region of the lesion, straining and dissecting the healthier side of the blood vessel. Moreover, in the case of nodular calcium, expansion of a standard angioplasty balloon can lead to pushing the node of calcified material in a manner that may puncture the vessel.

Another approach to dealing with calcified stenotic plaque is to cut away at a calcified lesion, by using a cutting or scoring balloon, an angioplasty balloon having a raised structure on the surface of the balloon (e.g., an angioplasty balloon with blade-like structures on its exterior). The expansion of an angioplasty balloon having a raised structure may allow for mechanical force on a lesion to be focused at the location of the raised structure, but these devices still do not provide for any protection from dissection or perforation resulting from preferential expansion of the balloon away from hardened tissue. Another technique for cutting away at a calcified lesion is by using an atherectomy device, which typically includes a motor-driven rotating or oscillating blade that is pushed into and cuts through an occlusion (also referred to as “debulking” or “extirpation”). Because these treatments work by liberating the calcified tissues from a blood vessel wall, there is an increased risk of embolism where the free-floating masses of calcification may proceed down the blood stream. Such systems may include baskets to capture or negative-pressure lumens to aspirate such unmoored emboli as a necessary additional structure to ensure the safety of such devices. An additional concern for atherectomy devices is that the movement or rotation of atherectomy catheter blades generates frictional heat and can cause a related thermal injury from mere operation of the atherectomy device. That heat can directly injure the lining of a blood vessel and can also lead to an increased risk of blood clotting. Naturally, the action of a moving blade within the vasculature also significantly increases the potential for a large dissection and perforation of the blood vessel by the blade itself.

Accordingly, there is an ongoing need for improved medical devices and treatments to address calcification and restore organ function. One such treatment is intravascular lithotripsy (IVL), which uses acoustic pressure to break up the calcified regions. In IVL, a device such as a catheter is advanced within the patient's body to a position adjacent to the treatment area. The IVL device is configured to generate acoustic waves, specifically, ultrasonic short pulse waves (also known as “shock waves”), which propagate outward from the IVL device to modify the calcified regions. The acoustic pressure of the shock waves may crack and disrupt the calcified regions near the IVL device without harming the surrounding blood vessels, tissues, or other organs. In particular, IVL can address and treat calcified plaques and stenosis with a safety profile that minimizes risk of blood vessel damage and with an efficacy profile that provides for durable circulatory restoration.

However, known IVL devices may not be as effective for treating lesions of larger body lumina, such as calcifications in the structural heart. Since sonic output from a shock wave emitter decays rapidly as the sonic energy propagates away from the emitter, to treat such anatomies, durable and low-profile catheters capable of generating high sonic output and/or positioning emitters closer to the target tissue is necessary.

### SUMMARY

According to aspects of the disclosure, systems, devices, and methods include catheter configurations for positioning shock wave emitters near lesions in relatively large body lumens, such as lesions in cardiac valves. Exemplary catheters include an emitter support member configured to position shock wave emitters in an annular loop (e.g., a “halo”) within a body lumen. The annular loop may be configured distribute the emitters around at least a portion of an annulus or leaflets of a cardiac valve.

According to aspects of the disclosure, shock wave emitter configurations improve shock wave emitter sonic output. For example, a shock wave emitter may include a conductive emitter band having an elongate slot that forms an electrode of the shock wave emitter. The “slotted” band may have improved sonic output relative to emitter bands with circular holes.

According to aspects of the disclosure, shock wave emitter configurations increase longevity of the shock wave emitters. The longevity of a shock wave emitter formed by an electrode pair may be improved by using a relatively larger and/or more durable electrode of the electrode pair as the anode of the electrode pair, which experiences relatively higher stresses than the cathode.

According to aspects of the disclosure, a catheter for treating calcified tissue in a body with shock waves includes: an elongate shaft extending from a proximal region to a distal region of the catheter and including a fluid lumen and a central axis; an enclosure located at the distal region of the catheter, the enclosure in fluid communication with a fluid source via the fluid lumen; a plurality of shock wave emitters enclosed within the enclosure and radially offset from the central axis, each of the shock wave emitters comprising at least one electrode pair configured to emit at least one shock wave radially outward from the central axis when a voltage pulse is applied across the electrode pair; and a conductive member electrically connected to the plurality of shock wave emitters from a voltage pulse generator.

In some embodiments, the enclosure is configured to be filled with a conductivity fluid to a pressure less than 5 atm. In some embodiments, the enclosure is a balloon. In some embodiments, the emitter includes a conductive band and each electrode pair includes a first electrode and a second electrode, the first electrode formed, at least in part, by a conductive surface of the conductive band, the conductive surface located along an elongate slot extending circumferentially along the conductive band. In some embodiments, the elongate slot extends 30 degrees to 330 degrees around a circumference of the conductive band. In some embodiments, the elongate slot extends 60 degrees to 120 degrees around a circumference of the conductive band. In some embodiments, the second electrode includes a conductive surface radially inward of the conductive band that is electrically connected to the conductive member. In some embodiments, the conductive surface is located on an inner band in contact with the conductive member. In some embodiments, the conductive surface is located on a distal region of the conductive member. In some embodiments, the plurality of shock wave emitters are connected to each other in series and the catheter includes a return wire that is electrically connected to the voltage pulse generator.

In some embodiments, the at least one electrode pair includes a first electrode having a first surface area and a second electrode having a second surface area greater than the first surface area. In some embodiments, the first electrode is configured to act as a cathode and the second electrode is configured to act as an anode. In some embodiments, the emitter includes a conductive band having an aperture, and the at least one electrode pair includes a first electrode formed by a distal surface of the conductive member and a second electrode formed by a conductive surface defined by the conductive band. In some embodiments, the enclosure includes an expanded diameter of 10 mm to 50 mm. In some embodiments, the enclosure, in an expanded state, includes a diameter greater than an axial length. In some embodiments, the enclosure includes a guidewire lumen. In some embodiments, the enclosure includes a disc-shape and the plurality of shock wave emitters are spaced within the enclosure. In some embodiments, the shock wave emitters are evenly spaced within the enclosure. In some embodiments, the enclosure includes a cylindrical coil shape defining an opening therethrough for blood to flow. In some embodiments, the shock wave emitters are mounted along a distal region of an elongate member that, in a first configuration, is received within the elongate shaft and, in a second configuration, is positioned within the enclosure. In some embodiments, the elongate member includes, at its distal region a shape memory material. In some embodiments, the shape memory material comprises nitinol. In some embodiments, the shock wave emitters are mounted along an elongate member that is slidably received in the elongate shaft.

According to aspects of the disclosure, a catheter system for treating calcified tissue in a body with shock waves includes: a voltage pulse generator; and a catheter including: a first conductive member; a second conductive member; a third conductive member; a first electrode pair including a first cathode and a first anode, where the first anode is formed from a more durable material than the first cathode; a second electrode pair including a second cathode and a second anode, where the second anode is formed from a more durable material than the second cathode. In some embodiments, current travels via the first conductive member from the voltage pulse generator to the first cathode, arcs from the first cathode to the first anode generating a first shock wave, travels via the second conductive member to the second cathode to the second anode generating a second shock wave, and returns via the third conductive member to the voltage pulse generator. In some embodiments, the first electrode pair and the second electrode pair are circumferentially distributed about a central axis of the catheter and the first electrode pair and the second electrode pair are configured such that the first and second shock waves propagate radially away from the central axis.

According to aspects of the disclosure, a catheter for treating calcified tissue in a body with shock waves includes: an emitter band having a slot that extends 30 degrees to 330 degrees around a circumference of the emitter band, where a conductive surface of the slot forms at least a part of at least a first electrode of an electrode pair; a conductive member having a conductive surface that forms at least a part of a second electrode of the electrode pair, where the catheter is configured to generate a shock wave when a voltage pulse is applied across the electrode pair; and an enclosure that is fillable with fluid. In some embodiments, the slot includes two slots. In some embodiments, the catheter includes a central axis and the electrode pair is configured such that the shock wave propagates radially away from the central axis. According to aspects of the disclosure, a catheter for treating calcified tissue in a body includes: an elongate shaft; and at least two shock wave emitters that, in a first configuration are received in the elongate shaft and, in a second configuration, are outside of the elongate shaft and spaced away from a longitudinal central axis of the elongate shaft.

According to aspects of the disclosure, a shock wave emitter band for a shock wave catheter includes a body and a slot that extends circumferentially around at least part of the body.

According to aspects of the disclosure, an electro-hydraulic method of generating shock waves includes: delivering a voltage pulse to a catheter, where the voltage pulse is delivered via a first conductive member that includes a first conductive surface spaced from a first emitter band by a first gap, where when the voltage pulse is applied across the first gap, a first shock wave is generated, where after the voltage pulse is applied across the first gap, the voltage pulse is delivered to a second emitter band by a second conductive member, which is in contact with the first emitter band and includes a second conductive surface spaced from the second emitter band by a second gap, where when the voltage pulse is applied across the second gap, a second shock wave is generated. In some embodiments, the calcified tissue to be treated with shock waves is located in the heart. In some embodiments, the calcified tissue to be treated with shock waves is at or proximate an aortic valve.

According to aspects of the disclosure, a catheter for generating shock waves comprises: a catheter body; a conductive band mounted to the catheter body, wherein the conductive band comprises at least one elongate slot that extends at least partially around the circumference of the conductive band; and at least one electrode positioned radially inward of the conductive band and aligned with the at least one elongate slot, wherein the at least one electrode and the conductive band form an electrode pair of a shock wave emitter.

Optionally, the elongate slot extends more than 180 degrees around the circumference of the conductive band.

Optionally, the elongate slot extends helically around the conductive band.

Optionally, a first electrode of the at least one electrode is positioned proximally of a second electrode of the at least one electrode, and wherein each of the first electrode and the second electrode are aligned with the elongate slot.

Optionally, the first electrode is spaced apart from the second electrode around the circumference of the conductive band by 180 degrees.

Optionally, the catheter includes an insulating layer positioned between the conductive band and the at least one electrode, wherein the insulating layer comprises at least one elongate slot aligned with the at least one elongate slot of the conductive band.

According to aspects of the disclosure, a catheter for generating shock waves comprises: a catheter body; a conductive band mounted to the catheter body, wherein a plurality of holes are formed into the conductive band at a plurality of circumferential locations of the conductive band; a conductive member positioned radially inward of the conductive band, wherein: a first portion of the conductive member is aligned with a first hole of the plurality of holes, wherein the first portion of the conductive member and the conductive band form an electrode pair of a first shock wave emitter; and a second portion of the conductive member is aligned with a second hole of the plurality of holes, wherein the second portion of the conductive member and the conductive band form an electrode pair of a second shock wave emitter.

Optionally, the conductive band comprises at least three holes and the conductive member is aligned with the at least three holes, thereby forming at least three shock wave emitters.

Optionally, the catheter includes a second conductive member positioned radially inward of the conductive band and spaced apart from the conductive member.

Optionally, a first end of the conductive member is spaced apart from a first end of the second conductive member by between 5 degrees and 45 degrees around the circumference of the conductive band.

Optionally, a second end of the conductive member is spaced apart from a second end of the second conductive member by between 5 degrees and 45 degrees around the circumference of the conductive band.

Optionally, the first conductive member and the second conductive member are semi-cylindrical.

Optionally, a first portion of the second conductive member is aligned with a third hole of the plurality of holes, wherein the first portion of the second conductive member and the conductive band form an electrode pair of a third shock wave emitter; and a second portion of the second conductive member is aligned with a fourth hole of the plurality of holes, wherein the second portion of the second conductive member and the conductive band form an electrode pair of a fourth shock wave emitter.

Optionally, the conductive member is aligned with a first three holes of the plurality of holes, thereby forming at least three shock wave emitters, and the second conductive member is aligned with a second three holes of the plurality of holes, thereby forming at least three different shock wave emitters.

Optionally, the second hole is positioned less than 180 degrees apart from the first hole around the circumference of the conductive band.

In some implementations, shock wave treatment of heart valve anatomy can be used as a preparatory procedure, to optimize the tissue region for receipt and implantation of a replacement heart valve.

According to some aspects, a catheter for generating shock waves comprises: a catheter body; an emitter support member configured to extend at least partially distally of a distal end of the catheter body, where the emitter support member comprises a pre-formed distal portion, where the pre-formed distal portion is configured to coil into an annular loop around at least a portion of a longitudinal axis of the catheter body; a plurality of shock wave emitters mounted to the emitter support member; and an enclosure enclosing the plurality of shock wave emitters.

Optionally, the enclosure comprises a preformed distal portion configured to coil into an annular loop around the longitudinal axis of the catheter body when filled with a conductive fluid. Optionally, the enclosure is configured to enable fluid flow through a body lumen. Optionally, the enclosure is configured to occlude fluid flow through a body lumen.

Optionally, the emitter support member is translatable between a deployed position and a retracted position. Optionally, in the retracted position, the pre-formed distal portion is not coiled. Optionally, the pre-formed distal portion of the emitter support member is deployable from an outer sheath. Optionally, in the deployed position, the plurality of shock wave emitters are circumferentially spaced from one another on the emitter support member around the longitudinal axis of the catheter body. Optionally, in the deployed position, the plurality of shock wave emitters are positioned at the same longitudinal location with respect to the catheter body. Optionally, at least one of the plurality of shock wave emitters comprises an emitter band mounted to the emitter support member wherein the emitter band comprises a slot that extends at least partially around the circumference of the emitter band. Optionally, the emitter band is oriented such that the slot faces in a distal direction relative to the catheter body when the pre-formed distal portion is coiled into the annular loop. Optionally, the emitter band is oriented such that the slot faces in a proximal direction relative to the catheter body when the pre-formed distal portion is coiled into the annular loop. Optionally, the emitter band is oriented such that the slot faces radially outward away from the longitudinal axis when the pre-formed distal portion is coiled into the annular loop. Optionally, each of the plurality of shock wave emitters comprises an emitter band, and each emitter band serves as an anode of an electrode pair that forms a respective shock wave emitter of the plurality of shock wave emitters. Optionally, the plurality of shock wave emitters are configured to generate shock waves biased in a particular direction and the plurality of shock wave emitters are positioned relatively closer to a portion of the enclosure that is opposite of the particular direction to prevent damage to the enclosure. Optionally, the pre-formed distal portion is configured to at least partially encircle a cardiac valve for treating calcified tissue proximate to the cardiac valve.

According to some aspects, a method of generating shock waves at a treatment site within a body lumen, the method comprising: positioning a distal portion of a catheter proximate a treatment site in a body lumen such that a plurality of shock wave emitters positioned at different locations along an emitter support member are adjacent different regions of the treatment site, where the emitter support member curves about a longitudinal axis of the catheter body such that the plurality of shock wave emitters are positioned a different circumferential positions about the longitudinal axis of the catheter body; and applying one or more energy pulses to the plurality of shock wave emitters to generate a plurality of shock waves.

Optionally, the plurality of shock wave emitters are spaced apart from one another along a length of the emitter support member. Optionally, the treatment site comprises a cardiac valve and the method comprises positioning the plurality of shock wave emitters adjacent to the cardiac valve. Optionally, the cardiac valve is an aortic valve, tricuspid valve, a mitral valve, or a pulmonary valve. Optionally, the plurality of shock wave emitters are oriented such that an electrode pair of at least a subset of the plurality of shock wave emitters is positioned adjacent to and facing a base of a valve leaflet. Optionally, the plurality of shock wave emitters are oriented such that an electrode pair of at least a subset of the plurality of shock wave emitters is positioned adjacent to and facing a valve annulus. Optionally, the method includes implanting an aortic valve implant after generating the plurality of shock waves. Optionally, the method includes positioning a valvuloplasty balloon at a valve annulus of the treatment site after generating the plurality of shock waves; and inflating the balloon to widen the valve annulus. Optionally, the method includes repositioning the plurality of shock wave emitters to target a different portion of the treatment site; and generating an additional plurality of shock waves. Optionally, the treatment site comprises a sub-annular valve. Optionally, the annular loop of the emitter support member is a closed loop. Optionally, an enclosure enclosing the annular loop of the emitter support member forms a closed loop. Optionally, the plurality of shock wave emitters are oriented such that sonic output produced during shock wave generation is directed in a primarily proximal direction, and wherein advancing the catheter to the treatment site comprises advancing the plurality of shock wave emitters across a valve annulus such that the treatment site is positioned proximally of the plurality of shock wave emitters.

According to some aspects, a catheter for generating shock waves comprises: a catheter body; a conductive band mounted to the catheter body, where the conductive band comprises at least one elongate slot that extends at least partially around the circumference of the conductive band; and at least one electrode positioned radially inward of the conductive band and aligned with the at least one elongate slot, where the at least one electrode and the conductive band form an electrode pair of a shock wave emitter.

Optionally, the at least one elongate slot extends at least 60 degrees around the circumference of the conductive band. Optionally, the elongate slot extends at least 180 degrees around the circumference of the conductive band. Optionally, the catheter includes at least one other electrode positioned radially inward of the conductive band and aligned with the at least one elongate slot. Optionally, the at least one other electrode and the conductive band form a second electrode pair, wherein the first and second electrode pairs are configured such that a voltage pulse applied to the first and second electrode pairs results in at least a first shock wave generated by the first electrode pair and at least a second shock wave generated by the second electrode pair. Optionally, the elongate slot extends helically around the conductive band. Optionally, a first electrode of the at least one electrode is positioned proximally of a second electrode of the at least one electrode, and wherein each of the first electrode and the second electrode are aligned with the elongate slot. Optionally, the first electrode is spaced apart from the second electrode around the circumference of the conductive band by 180 degrees. Optionally, the at least one elongate slot comprises an elongate slot spaced apart from the at least one elongate slot, wherein the elongate slot extends at least partially around the circumference of the conductive emitter band. Optionally, the catheter includes an insulating layer positioned between the conductive band and the at least one electrode, wherein the insulating layer comprises at least one elongate slot aligned with the at least one elongate slot of the conductive band.

According to some aspects, a catheter for treating calcified tissue in a body with shock waves comprises: a first electrode pair and a second electrode pair, each of the first and second electrode pairs comprising a cathode and an anode, where the anode is formed from a more durable material than the cathode, where an anode of the first electrode pair is connected to a cathode of the second electrode pair; a pair of conductors electrically connected to the first and second electrode pairs; where the catheter is configured such that a voltage pulse applied across the pair of conductors causes current to arc from a cathode of the first electrode pair to the anode of the first electrode pair, thereby generating a first shock wave, and to arc from the cathode of the second electrode pair to an anode of the second electrode pair, thereby generating a second shock wave.

Optionally, the first electrode pair and the second electrode pair are circumferentially distributed about a longitudinal axis of the catheter and the first electrode pair and the second electrode pair are configured such that the first and second shock waves propagate radially away from the longitudinal axis. Optionally, each anode of a respective pair of electrodes has a greater surface area than each cathode of the respective pair of electrodes. Optionally, the anode of each of the first and second pairs of electrodes is formed by a conductive band.

According to some aspects, a catheter for generating shock waves comprises: a catheter body; a conductive band mounted to the catheter body, where the conductive band comprises a plurality of holes at a plurality of circumferential locations of the conductive band; and a conductive member positioned radially inward of the conductive band, where: a first portion of the conductive member is aligned with a first hole of the plurality of holes, where the first portion of the conductive member and the conductive band form an electrode pair of a first shock wave emitter, and a second portion of the conductive member is aligned with a second hole of the plurality of holes, where the second portion of the conductive member and the conductive band form an electrode pair of a second shock wave emitter.

Optionally, the conductive band comprises at least three holes and the conductive member is aligned with the at least three holes, thereby forming at least three shock wave emitters. Optionally, the catheter comprises a second conductive member positioned radially inward of the conductive band and spaced apart from the conductive member. Optionally, an end of the conductive member is spaced apart from an end of the second conductive member by between 5 degrees and 45 degrees around the circumference of the conductive band. Optionally, the first conductive member and the second conductive member are semi-cylindrical.

According to some aspects, a catheter comprises: an outer sheath; an elongate member positioned at least partially within the outer sheath; a plurality of shock wave emitters mounted to a distal portion of the elongate member; an inner member positioned at least partially within the elongate member, wherein the inner member comprises a pre-formed distal portion configured such that in a deployed position distal of a distal end of the outer sheath, the pre-formed distal portion unfolds into an annular loop around at least a portion of a longitudinal axis of the catheter body. Optionally, the elongate member is configured to translate along the inner member and conform to the loop of the pre-formed distal portion to position the plurality of shock wave emitters along the loop. Optionally, the elongate member is bonded to the pre-formed distal portion of the inner member, such that in the deployed configuration, the elongate member unfolds into the loop and the plurality of shock wave emitters are positioned along the loop. Optionally, the pre-formed distal portion is configured such that in a retracted position within the outer sheath, the pre-formed distal portion folds into a folded configuration. Optionally, the catheter comprises: an enclosure sealed to a distal end of the elongate member or the inner member.

In some embodiments, any one or more of the characteristics of any one or more of the systems, methods, and/or computer-readable storage mediums recited above may be combined, in whole or in part, with one another and/or with any other features or characteristics described elsewhere herein.

### BRIEF DESCRIPTION OF THE FIGURES

The invention will now be described, by way of example only, with reference to the accompanying drawings, in which:

FIG. 1 illustrates an exemplary shock wave catheter having an occluding balloon and circumferentially-distributed shock wave emitters according to some examples.

FIG. 1B illustrates a detail view of a shock wave emitter according to some examples.

FIG. 2 illustrates an exemplary shock wave catheter having a non-occluding balloon and circumferentially-distributed shock wave emitters according to some examples.

FIG. 3A illustrates a slotted emitter band according to some examples.

FIG. 3B illustrates another exemplary slotted emitter band according to some examples.

FIG. 3C illustrates another exemplary slotted emitter band according to some examples.

FIG. 3D illustrates another exemplary slotted emitter band according to some examples.

FIG. 3E illustrates an exemplary slotted emitter band forming two shock wave emitters according to some examples.

FIG. 3F illustrates an exemplary slotted emitter band forming one shock wave emitter according to some examples.

FIG. 3G illustrates relative sonic output generated using emitter bands with standard holes compared to slotted emitter bands according to some examples.

FIG. 4 illustrates a wiring schematic for a shock wave catheter according to some examples.

FIG. 5 illustrates another wiring schematic for a shock wave catheter according to some examples.

FIG. 6 illustrates an exemplary directional arcing schematic according to some examples.

FIG. 7 illustrates another exemplary directional arcing schematic according to some examples.

FIG. 8 illustrates an exemplary computing system according to some examples.

FIG. 9 illustrates an exemplary shock wave catheter system according to some examples.

FIG. 10A illustrates an exemplary shock wave emitter assembly according to some examples.

FIG. 10B illustrates the exemplary shock wave emitter assembly of FIG. 10A according to some examples.

FIG. 11A illustrates another exemplary shock wave emitter assembly according to some examples.

FIG. 11B illustrates the exemplary shock wave emitter assembly of FIG. 11A according to some examples.

FIG. 12A illustrates another exemplary shock wave emitter assembly according to some examples.

FIG. 12B illustrates the exemplary shock wave emitter assembly of FIG. 12A according to some examples.

FIG. 12C shows additional detail of the inner electrodes that may be included in the shock wave emitter assembly of FIG. 12A according to some examples.

FIG. 13 illustrates an exemplary catheter having an emitter support member configured to circumferentially-distribute shock wave emitters according to some examples.

FIGS. 14A-14C illustrate an exemplary catheter positioned adjacent an aortic valve according to some examples.

FIG. 15 illustrates an exemplary method for generating shock waves in a body lumen according to some examples.

FIG. 16 illustrates an exemplary catheter with distally biased shock wave emitters positioned adjacent a cardiac valve.

FIG. 17 illustrates an exemplary catheter with radially biased shock wave emitters positioned adjacent a cardiac valve.

FIG. 18 illustrates an exemplary catheter with proximally biased shock wave emitters positioned adjacent a cardiac valve according to some examples.

FIG. 19 illustrates an exemplary catheter with radially inwardly biased shock wave emitters positioned adjacent a cardiac valve according to some examples.

FIGS. 20A-20E illustrate aspects of an exemplary catheter including an emitter support member configured to be translated over a pre-formed inner member according to some examples.

### DETAILED DESCRIPTION

The following description is presented to enable a person of ordinary skill in the art to make and use the various embodiments and aspects thereof disclosed herein. Descriptions of specific catheters, systems, methods, and applications are provided only as examples. Various modifications to the examples described herein will be readily apparent to those of ordinary skill in the art, and the general principles described herein may be applied to other examples and applications without departing from the spirit and scope of the various embodiments and aspects thereof. Thus, the various embodiments and aspects thereof are not intended to be limited to the examples described herein and shown but are to be accorded the scope consistent with the claims.

In recent years, in order to treat atherosclerosis and related conditions, the technique and treatment of intravascular lithotripsy (“IVL”) has been developed, which is an interventional procedure to modify calcified plaque in diseased vasculature. More precisely, IVL is the energy-based generation of ultrasonic acoustic pressure waves for modification, fracture, and fragmentation of vascular calcification in situ. The mechanism of plaque modification is through use of a catheter having one or more ultrasonic short pressure pulses (commonly referred to as “shock waves”) emit from a generating source located within a liquid that can create acoustic ultrasonic shock waves that modify and fracture the calcified plaque. IVL improves arterial compliance and enables optimal lumen expansion in vascular interventional procedures. IVL devices vary in design with respect to the energy source used to generate the acoustic shock waves, with two exemplary energy sources being electrohydraulic generation and laser generation. Moreover, the broader application of intracorporeal lithotripsy, leveraging the systems and techniques of IVL, can be used for treatment of other tissues and organs within a patient's body, as in the present disclosure for the treatment of structural heart anatomy.

For electrohydraulic generation of ultrasonic short pressure pulses, a conductive solution (e.g., saline) can be contained within an enclosure that surrounds electrodes or can be flushed through a tube that surrounds the electrodes. The calcified plaque modification is achieved by creating ultrasonic shock waves within the catheter by an electrical discharge (e.g., a plasma arc) across the electrodes. The energy from this electrical discharge enters the surrounding fluid, generating an acoustic shock wave where the wave itself is ultrasonic (i.e., a wave that has frequency components of greater than 20,000 Hz). In addition, the discharge creates one or more rapidly expanding and collapsing vapor bubbles that generate secondary shock waves due to the cavitation of the collapsing vapor bubble. The shock waves propagate radially outward and modify calcified plaque within the blood vessels. The shock waves travel deeply and safely through soft arterial tissue because of the acoustic impedance soft tissue, which is similar to water. Acoustic impedance is a function of the density and the elasticity of a material and the speed of sound through that material. When the shock waves encounter tissues with a different acoustic impedance, such as intimal calcification of plaque close to the surface or endothelium of a vessel or medical calcification in the smooth muscle layer of a vessel, the leading edge of the shock wave imparts compressive stress on the calcified tissue. Shearing occurs on the lesion as the shock wave passes through the calcification. When the shock wave reaches the distal boundary of the calcification, the shock wave is both transmitted and reflected, inducing tensile stress that pulls the calcification apart. Further compressive stress is applied by the squeezing which occurs when the ultrasonic shock wave entering the calcium propagates faster than the remaining shock wave travelling outside the calcified region of tissue. These forces generated by IVL result in multi-plane and longitudinal fractures of the calcification in the tissue.

More specifically, catheters to deliver IVL therapy have been developed that include pairs of electrodes for electrohydraulically generating shock waves inside an angioplasty balloon. Shock wave devices can be particularly effective for treating calcified plaque lesions because the acoustic pressure from the shock waves can crack and disrupt lesions near the angioplasty balloon without harming the surrounding tissue. In these devices, the catheter is advanced over a guidewire through a patient's vasculature until it is positioned proximal to and/or aligned with a calcified plaque lesion in a body lumen. The balloon is then inflated with conductive fluid (e.g., using a relatively low pressure of 2-4 atm) so that the balloon expands to contact the lesion but not to a degree that substantively displaces the lesion. Voltage pulses can then be applied across the electrodes of electrode pairs to produce acoustic shock waves that propagate through the walls of the angioplasty balloon and into the lesions. Once the lesions have been cracked by the acoustic shock waves, the balloon can be expanded further to increase the cross-sectional area of the lumen and improve blood flow through the lumen. Alternative devices to deliver IVL therapy can include electrodes disposed within a closed volume other than an angioplasty balloon, such as a cap, balloons of variable compliancy, or other type of enclosure.

Critically, the calcified plaque remains in place following the shock waves; for IVL intimal calcium remains in the blood vessel lining and medical calcium remains in the muscle tissue surrounding the blood vessel. IVL generally does not cause the debulking or extirpation of tissue from a blood vessel wall. Similarly, for calcification of structural heart tissues such as organ walls, arteries and veins, valve leaflets, commissures, and the like, calcification that is fragmented by ultrasonic short pressure pulses does not separate from the surface of the lesion, but remains within the target tissue while the target tissue has become more pliable and flexible.

Accordingly, the IVL process can also be considered different from standard atherectomy procedures and different from cutting or scoring balloons at least in that IVL cracks calcium but does not liberate the calcium from the tissue. Hence, generally speaking, IVL systems should not require aspiration nor embolic protection. Accordingly, IVL does not carry the same degree of risk of embolism, perforation, dissection, or other damage to vasculature as atherectomy procedures or angioplasty procedures using cutting or scoring balloons. In further contrast with cutting techniques, due to the compliance of a normal blood vessel and non-calcified plaque, the shock waves produced by IVL do not modify the normal healthy vessel tissue or non-calcified plaque. In other words, the shock waves from IVL do not have an adverse clinical impact on soft tissues while treating the hardened calcified anatomy.

For laser generation of acoustic shock waves, a laser pulse is transmitted into and energy from the laser is absorbed by a fluid within the catheter, optionally with a target to act as catalyst for the laser absorption. This absorption process rapidly heats and vaporizes the fluid, thereby generating the rapidly expanding and collapsing vapor bubble, as well as the acoustic shock waves that propagate outward and modify the calcified plaque. The acoustic shock wave intensity is higher if a fluid is chosen that exhibits strong absorption at the laser wavelength that is employed. These examples of electrohydraulic and laser-based IVL devices are not intended to be a comprehensive list of potential energy sources to create the ultrasonic IVL shock waves.

Examples disclosed herein include systems, methods, and devices for positioning shock wave emitters in close proximity to lesions within a body lumen. An exemplary shock wave catheter disclosed herein may include an emitter support member carrying a plurality of shock wave emitters. The emitter support member may include a pre-formed distal portion that is configured to coil into an annular loop, or “halo.” The pre-formed distal portion may coil into an annular loop around a longitudinal axis of the catheter body such that the loop is substantially concentric with the catheter body and with an inner circumference of a body lumen. The annular loop configuration may correspond to the shape of a valve annulus, for instance, an aortic or mitral valve annulus. Thus, the annular loop configuration may enable the plurality of shock wave emitters carried by the emitter support member to be positioned at a plurality of different locations around the annulus and/or leaflets of a valve.

According to some aspects, systems, devices, and methods are disclosed herein for enhancing sonic output relative to conventional shock wave emitters. Exemplary shock wave emitters may include emitter bands having one or more elongate slots formed into the emitter band and extending at least partially around the circumference of the emitter band. At least one electrode may be positioned radially inward of the conductive band and aligned with the elongate slot to form a shock wave emitter. Compared with conventional emitter bands that employ discrete apertures (e.g., circular holes), the elongate-slot emitter band architecture provides increased sonic output, thus improving the therapeutic effect of the shock wave emitters disclosed herein.

According to some aspects, systems, devices, and methods disclosed herein enable extended longevity of electrodes used to form shock wave emitters. According to some examples, disclosed herein are shock wave catheters including a plurality of shock wave emitters. Each emitter may include an electrode pair—an anode and a cathode—spaced apart by a spark gap. The anode of each electrode pair may be configured to be relatively more durable than the cathode of each electrode pair. For instance, the anode of each electrode pair may be an emitter band while the cathode of each electrode pair may be a wire end. The anode of a shock wave emitter (i.e., the electrode that receives an arcing current from a cathode) experiences higher stresses during shock wave generation. Using a relatively more durable electrode for the anode of each emitter may promote shock wave emitter longevity and uniform electrode degradation.

As a form of therapy, shock wave treatment of heart valve anatomy can be used as a preparatory procedure, to optimize the tissue region for receipt and implantation of a replacement heart valve. More specifically, heart valve implants, both mechanical and tissue-based, can be challenging to implant and seat on the target location where the target tissue is hardened due to calcification. The application of ultrasonic short pressure pulses prior to a replacement valve implantation procedure can make the target location more pliable and flexible, thereby allowing an implant to more easily access and orient at the implant location. Moreover, the relatively pliable and flexible heart valve tissue can allow for the implant to securely sit or anchor at and around the annular region of the valve, thereby forming a better seal at the perimeter of the valve implant, reducing any seepage of leakage of fluid going around the main passage of the valve implant. Accordingly, the application of intracorporeal lithotripsy to a heart valve can provide for improved performance and longevity of a valve implant.

Notably, ultrasonic short pressure pulses have a non-ablative mechanism of action, unlike other structural heart therapies such as radiofrequency ablation (heat-based) or cryoablation. Of course, ultrasonic short pressure pulses delivered to structural heart tissues can be used in combination with ablation-based therapies as appropriate.

As provided herein, it should be appreciated that any disclosure of a numerical range describing dimensions or measurements such as thicknesses, length, weight, time, frequency, temperature, voltage, current, angle, etc. is inclusive of any numerical increment or gradient within the ranges set forth relative to the given dimension or measurement. It should be further appreciated that any disclosure of a numerical range as a boundary term or inequality term is similarly inclusive of any numerical increment or gradation within the given range; e.g., recitation of a parameter that is “at least a defined value, where the defined value ranges from 5% to 50%” supports the disclosure of that parameter being “at least 5%”, “at least 50%”, “at least 37%”, “at least 42.4%”, and the like. Furthermore, numerical designators such as “first,” “second,” “third,” “fourth,” etc. are merely descriptive and do not indicate a relative order, location, or identity of elements or features described by the designators. For instance, a “first” shock wave may be immediately succeeded by a “third” shock wave, which is then succeeded by a “second” shock wave. As another example, a “third” emitter may be used to generate a “first” shock wave and vice versa. Accordingly, numerical designators of various elements and features are not intended to limit the disclosure and may be modified and interchanged.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof. As provided herein, it should be appreciated that any disclosure of a numerical range describing dimensions or measurements such as thicknesses, length, weight, time, frequency, temperature, voltage, current, angle, etc. is inclusive of any numerical increment or gradient within the ranges set forth relative to the given dimension or measurement.

As used herein, the term “electrode” refers to an electrically conducting element (typically made of metal) that receives electrical current and subsequently releases the electrical current to another electrically conducting element. In the context of the present disclosure, electrodes are often positioned relative to each other, such as in an arrangement of an inner electrode and an outer electrode. Accordingly, as used herein, the term “electrode pair” refers to two electrodes that are positioned adjacent to each other such that application of a sufficiently high voltage to the electrode pair will cause an electrical current to transmit across the gap (also referred to as a “spark gap”) between the two electrodes (e.g., from an inner electrode to an outer electrode, or vice versa, optionally with the electricity passing through a conductive fluid or gas therebetween). In some contexts, one or more electrode pairs may also be referred to as an electrode assembly. In the context of the present disclosure, the term “emitter” broadly refers to the region of an electrode assembly where the current transmits across the electrode pair, generating a shock wave. The term “emitter sheath” or “emitter band” (which are used interchangeably) refers to a sheath/band of conductive material that may form one or more electrodes of one or more electrode pairs, thereby forming a location of one or more emitters.

Components of emitters, including electrodes and emitter sheaths/bands, may be formed from a metal, such as stainless steel, copper, tungsten, platinum, palladium, molybdenum, cobalt, chromium, iridium, an alloy or alloys thereof, such as cobalt-chromium, platinum-chromium, cobalt-chromium-platinum-palladium-iridium, or platinum-iridium, or a mixture of such materials.

For treatment of an occlusion in a blood vessel, the voltage pulse applied by a power source, including any of the power sources described herein (which may also be referred to herein as voltage sources or pulse generators), can be in the range of from about five hundred to three thousand volts (500 V-3,000 V). In some implementations, for the treatment of stenosis in a blood vessel or of another anatomical feature (e.g., structural heart tissues), the voltage pulse applied by the voltage source can be up to about fifteen thousand volts (15,000 V) or higher than fifteen thousand volts (15,000 V). The pulse width of the applied voltage pulses ranges between one microsecond and six microseconds (1-6 μs). The repetition rate or frequency of the applied voltage pulses may be between about 1 Hz and 10 Hz. The total number of pulses applied by the power source to a treatment device (e.g., an IVL catheter) can be, for example, sixty (60) pulses, eighty (80) pulses, one hundred twenty (120) pulses, three hundred (300) pulses, or up to five hundred (500) pulses, or any increments of pulses within this range. Further implementations of power sources can deliver greater than 500 pulses to a treatment device. Alternatively or additionally, in some examples the power source may be configured to deliver a packet of micro-pulses (e.g., 500 micro-pulses in a packet) having a sub-frequency between about 10 Hz-10 kHz. The preferred voltage, repetition rate, and number of pulses for any given IVL device or treatment may vary depending on factors such as the size, length, eccentricity, nodularity, or orientation of the lesion, the extent of lesion or tissue calcification, the size of the blood vessel, the attributes of the patient (e.g., age, gender, predisposition to cardiac disease, etc.), or the stage of treatment. In delivering a treatment regime, a physician may start with low energy shock waves and increase the energy as needed during the procedure, or vice versa. The amount of power delivered for shock waves may further vary during the course of a procedure, following a predetermined sequence of energy increases or decreases, or by changing the amount of energy delivered in response to sensor data obtained prior to and/or during the IVL treatment procedure. The magnitude of the shock waves can be controlled by controlling the voltage, current, duration, and repetition rate of the pulsed voltage from the power source.

In some implementations, an IVL catheter may be a “rapid exchange-type” (“RX”) catheter provided with an opening portion through which a guidewire can be guided (such as through a middle portion of a central tube in a longitudinal direction). In some other implementations, an IVL catheter may be an “over-the-wire-type” (“OTW”) catheter in which a guidewire lumen is formed throughout the overall length of the catheter, and a guidewire can be guided through the proximal end of a hub. A guidewire lumen entry point to a catheter is at or proximate to the distal end of the catheter tip, and the guidewire lumen extends through a portion of the catheter to an exit port. Thus in use, a guidewire is delivered into the anatomy of a patient, the proximal end of the guidewire (outside the patient) is fed into the distal end opening of the catheter, and the catheter is run along the guidewire until it reaches the target tissue at the distal end of the guidewire (inside the patient); the effective difference between an OTW and an Rx catheter is where the guidewire exits the catheter. The selection between an OTW design and an Rx design is driven by factors including (but not limited to): anatomy to be treated (e.g., coronary vasculature vs. peripheral vasculature); the length of guidewire to be used; the trackability, stiffness, torque transmission, and deliverability of the catheter; the profile and cross-section of the catheter, the ability to exchange a wire when the catheter is past a stenosis; positioning of the distal end of a catheter close to the end of a guidewire and further obtaining positional confirmation of the catheter.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the following description of the various embodiments, reference is made to the accompanying drawings, in which are shown, by way of illustration, specific embodiments that can be practiced. It is to be understood that other embodiments and examples can be practiced, and changes can be made without departing from the scope of the disclosure.

Efforts have been made to improve the design of electrode assemblies included in shock wave and directed cavitation catheters. For instance, low-profile electrode assemblies have been developed that reduce the crossing profile of a catheter and allow the catheter to more easily navigate calcified vessels to deliver shock waves in more severely occluded regions of vasculature. Examples of low-profile electrode designs can be found in U.S. Pat. Nos. 8,888,788, 9,433,428, 10,555,744, and 10,709,462, in U.S. Publication No. 2021/0085383, and in U.S. patent application Ser. No. 18/586,299, all of which are incorporated herein by reference in their entireties. Other catheter designs have improved the delivery of shock waves, for instance, by specific electrode construction and configuration thereby directing shock waves in a forward direction to break up tighter and harder-to-cross occlusions in vasculature. Examples of forward-biased or firing-firing catheter designs can be found in U.S. Pat. Nos. 10,966,737, 11,478,261, and 11,596,423, in U.S. Publication Nos. 2023/0107690 and 2023/0165598, and in U.S. patent application No. Ser. No. 18/524,575 and Ser. No. 18/680,853, all of which are incorporated herein by reference in their entireties. Efforts to increase device longevity have included pre-conditioning an electrode surface with a lower voltage pulse and alternating electrode polarity during pulsing as described U.S. Pat. Nos. 9,138,249 and 10,226,265, which are incorporated herein by reference in their entireties. Catheter systems have also been developed to control energy output from shock wave emitters as described in U.S. Pat. No. 9,333,000, which is incorporated herein by reference in its entirety.

According to aspects of the disclosure, a catheter for treating calcified tissue includes a pre-formed memory shape elongate member (e.g., a nitinol wire) configured in a 360-degree radial orientation when deployed. A sliding sheath or sliding wire or similar mechanism may allow for the pre-formed nitinol with emitters to be retracted into a straight configuration and deployed into its radial configuration when desired. Any number of emitters may be used for 360 degrees coverage or any other angles and shapes.

According to aspects of the disclosure, shock wave emitters are configured to generate shock waves that propagate radially outwards from a central axis of the catheter.

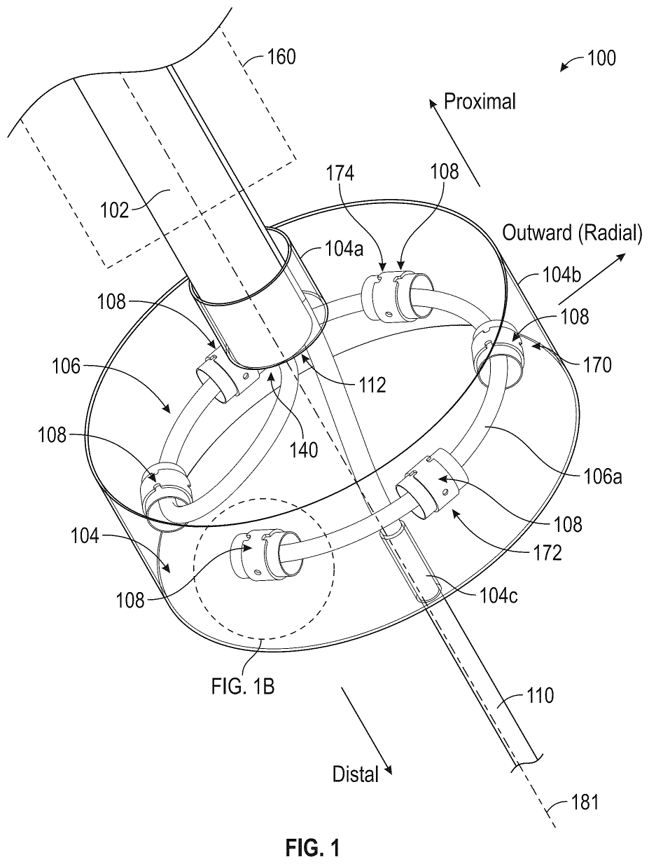

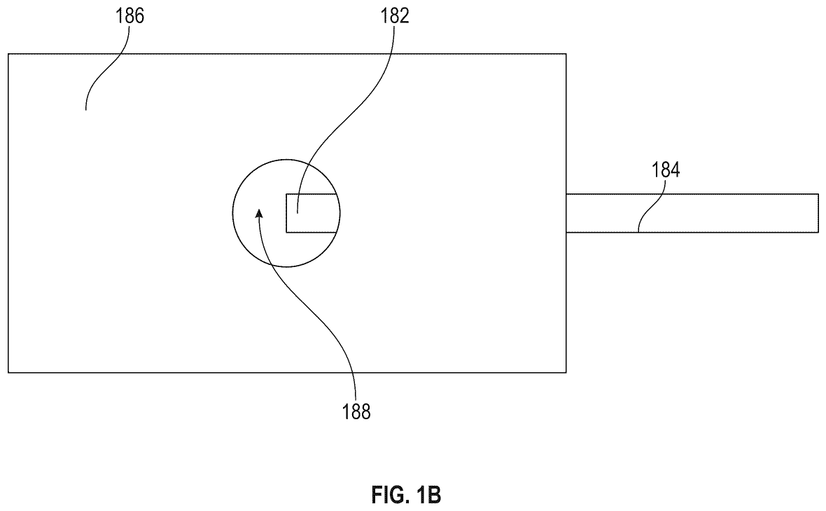

FIG. 1 illustrates aspects of an exemplary catheter 100 for treating lesions (e.g., occlusions, calcifications, etc.) in body lumens, such as within or near valves (e.g., cardiac valves). Catheter 100 may include a catheter body 102 (which may be an elongate sheath/elongate shaft) and an emitter support member 106. The emitter support member 106 may include a pre-formed distal portion 106a at its distal end that supports at least one shock wave emitter 108. The pre-formed distal portion 106a may be shaped so as to position the at least one shock wave emitter 108 further from a longitudinal axis 181 of the catheter body 102 and, thereby, closer to a lesion within a body lumen, particularly a lesion within a large body lumen such as a lesion in a cardiac valve.

A plurality of shock wave emitters 108 may be mounted to the emitter support member 106 along the pre-formed distal portion 106a. The pre-formed distal portion 106a may be configured to coil into an annular loop. The annular loop may loop around (e.g., circumscribe) the longitudinal axis 181 of the catheter body 102. In some examples, the annular loop formed by the pre-formed distal portion 106a is configured to conform to the shape and/or size of a valve, such as a mitral or aortic valve annulus. Accordingly, the plurality of shock wave emitters can be easily positioned at a plurality of different locations around the circumference of the valve for shock wave treatment.

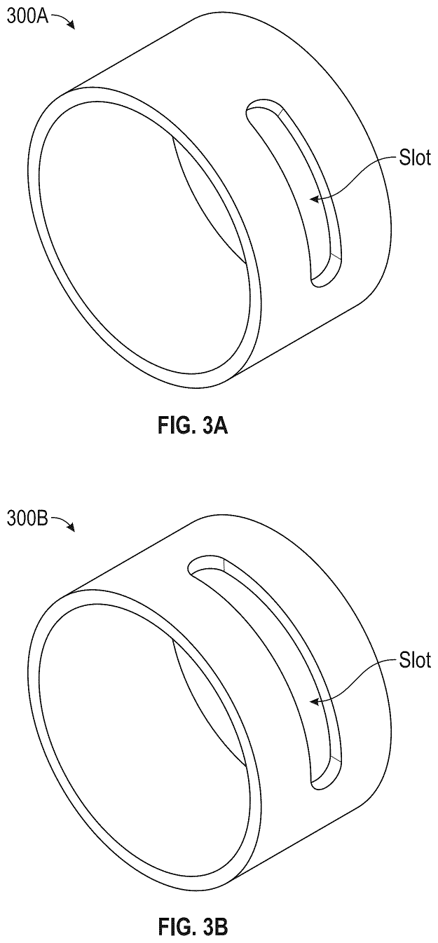

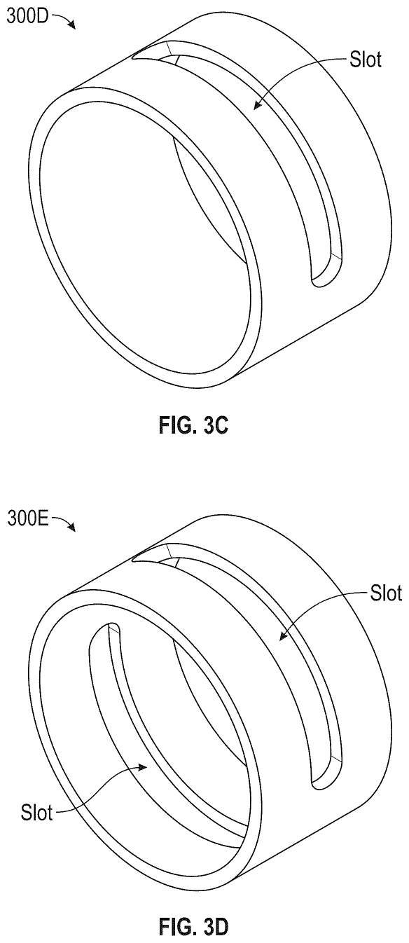

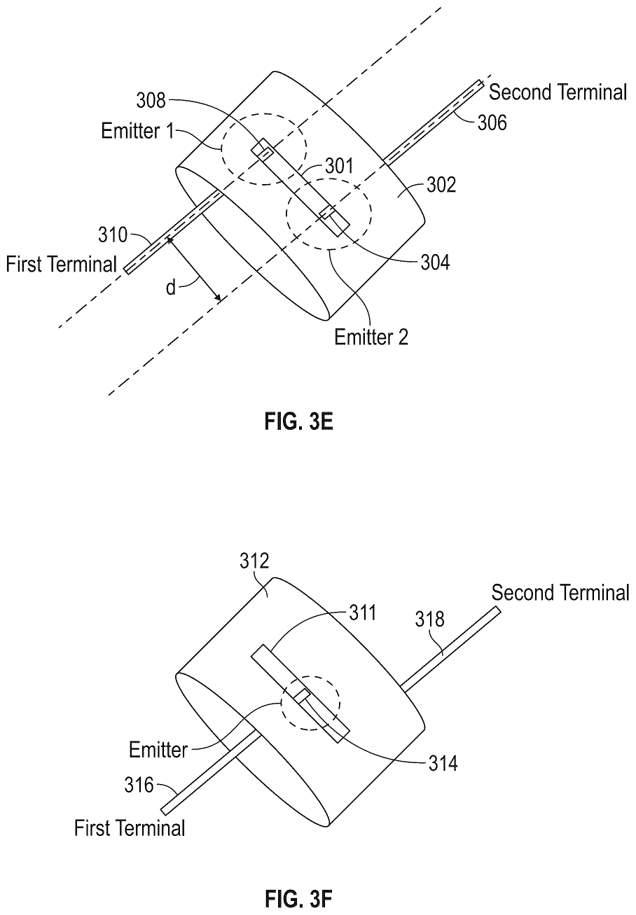

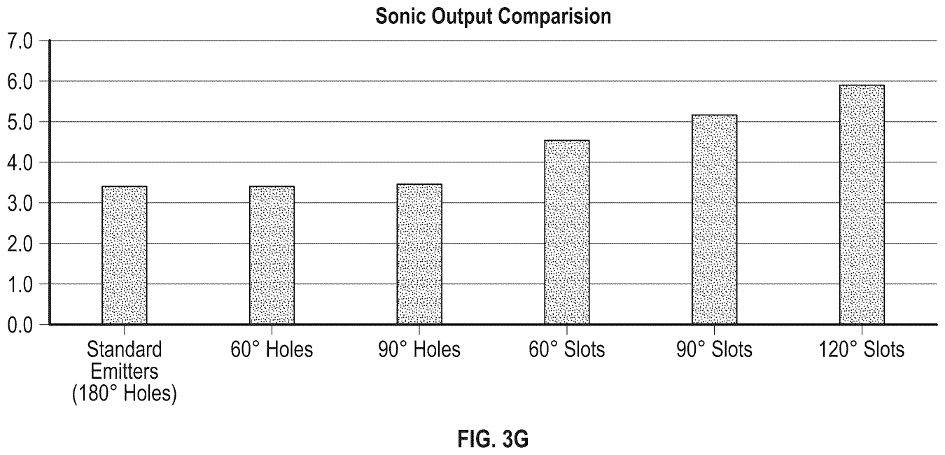

The plurality of shock wave emitters 108 may be positioned on the pre-formed distal portion 106a such that when the pre-formed distal portion 106a is deployed from the catheter body 102, the plurality of shock wave emitters 108 are spaced circumferentially from one another about a longitudinal axis 181 of catheter 100. As illustrated in the detail view shown in FIG. 1B, each of the plurality of shock wave emitters 108 may be formed by an electrode pair including a respective emitter sheath 186 positioned adjacent to and spaced apart from a conductor 182 (e.g., an end of a conductive wire 184) by a spark gap 188. One or more of the emitter sheaths 186 may form an electrode of a plurality of shock wave emitters, and a plurality of emitter sheaths 186 may be positioned on the pre-formed distal portion. It should be understood that other emitter configurations are usable for the device shown in FIG. 1, including but not limited to those shown in FIGS. 3E-7 and FIGS. 10A-12C. The plurality of shock wave emitters 108 may be positioned at the same longitudinal location as one another but circumferentially offset from one another, thus forming a circular array (often referred to herein as a “halo”) of emitters, as illustrated in the example of FIG. 1.

At least some of the electrode pairs of the plurality of shock wave emitters 108 may be positioned on a circumferential location 170 of the emitter support member 106 that faces radially outward (e.g., 180 degrees from the side of emitter support member 106 that faces inwardly toward longitudinal axis 181). Thus, shock waves generated using the at least one shock wave emitter 108 may be directed outward away from the longitudinal axis 181. In some examples, at least some of the electrode pairs of the plurality of shock wave emitters 108 may be positioned between 0 degrees and 180 degrees offset from the circumferential location of emitter support member 106 that faces longitudinal axis 181. For instance, at least some of the electrode pairs of the plurality of shock wave emitters 108 may be positioned at 90 degrees offset from the circumferential location of emitter support member 106 that faces longitudinal axis 181. Thus, shock waves generated using the at least one shock wave emitter 108 may be directed distally (e.g., when positioned on distal-facing circumferential location 172 of the emitter support member 106) or proximally (e.g., when positioned on proximal-facing circumferential location 174 of the emitter support member 106).

In some embodiments, a shock wave catheter (e.g., the catheter 100 shown in FIG. 1) includes an enclosure 104. The enclosure (e.g., enclosure 104) may be in the form of an occluding balloon (e.g., a balloon that obstructs fluid flow through a body lumen) having a diameter of 10 mm to 50 mm. In some embodiments, a shock wave catheter such as catheter 100 includes an occluding ballon, such as enclosure 104, having a diameter of 10 mm to 40 mm. In some embodiments, an occluding balloon, such as enclosure 104, has a disc shape. In some examples, the enclosure 104 has a diameter greater than an axial length when expanded/filled with a fluid. The enclosure 104 may be sealed to a distal end of the catheter body 102 via a proximal leg portion 104a that has a diameter smaller than an outer diameter 104b of the enclosure 104. In some embodiments, an occluding balloon, such as enclosure 104, may be used in the aortic and/or mitral space with a distally centered guidewire lumen 110 for over the wire access and deployment. The enclosure 104 may be fillable with a fluid (e.g., a conductive fluid). In some examples, a fluid lumen 140 is provided within the catheter body 102 and extends along the catheter body from the proximal end to the enclosure 140. The fluid lumen 140 may be configured to introduce and/or withdraw fluid from enclosure 104.

At least a portion of the guidewire lumen 110 may be positioned radially outward of the catheter body 102. The guidewire lumen 110 may extend (e.g., in parallel) alongside an outer surface of the catheter body 102 from a proximal end of the catheter body 102 to a distal end of the catheter body. A portion of the guidewire lumen 110 may angle radially inward toward the longitudinal axis 181 distally of a distal end of the catheter body 102 for a first distance and may turn to run parallel and/or concentrically with longitudinal axis 181 for a second distance distally of the distal end of the catheter body. A portion of the guidewire lumen 110 may positioned within the enclosure 104. The guidewire lumen 110 may enter the enclosure 104 via the proximal leg portion. A distal leg portion 104c of the enclosure 104 may be sealed to guidewire lumen 110, and the guidewire lumen 10 may exit the enclosure 104 via the distal leg portion 104c. In some examples, rather than being positioned radially outward of the catheter body, the guidewire lumen 10 may instead extend within or be formed by a portion of catheter body 102. The guidewire lumen 10 may be positioned radially within the catheter body. The guidewire lumen 10 may be radially offset from the emitter support member 106.

In some examples, the catheter body 102 and/or the emitter support member 106 are configured to be introduced into a body lumen via an outer sheath 160 (e.g., an introducer). The catheter body 103 and/or emitter support member 106 may be retractable into and/or deployable from the outer sheath 160. In some examples, the enclosure 104 is retractable into the outer sheath 160. An inner diameter of the outer sheath may be greater than an outermost diameter of the enclosure 104 when the enclosure 104 is inflated or filled with a fluid such that the enclosure 104 can be retracted into and extended out of a distal end of the outer sheath. In some examples, the emitter support member 106 is fixedly attached to the catheter body 102. In some examples, the emitter support member 106 may be configured such that it can be translated longitudinally relative to the catheter body 102. For instance, the emitter support member 106 may be retractable into the catheter body 102 such that when a user retracts the emitter support member 106 the pre-formed loop 106a portion is straightened as it is pulled into a distal opening 112 of the catheter body 102. The emitter support member 106 may likewise be deployable from a retracted position. A user may translate the emitter support member 106 distally relative to the catheter body 102 to deploy the pre-formed distal portion 106a from a distal opening 112 of the catheter body 102. When deployed from the distal opening 112, the pre-formed distal portion may naturally coil into the annular loop around a longitudinal axis 181 forming a “halo” shape, as illustrated in FIG. 1.

In some examples, enabling retraction of the emitter support member 106 into the catheter body 102 may improve the navigability of catheter 100. For instance, the emitter support member 106 may be retracted into the catheter body 102, and the enclosure 104 may be deflated or emptied of a conductive fluid, which may reduce the size of enclosure 104, thus reducing the overall profile of catheter 100. The catheter 100 may be inserted into a body lumen with the enclosure 104 deflated and advanced over a guidewire (extending through guidewire lumen 110) to a treatment site, such as an aortic or mitral valve. Once at the treatment site, the enclosure 104 may be filled or inflated with a conductive fluid, and then the emitter support member 106 may be deployed, as illustrated in FIG. 1. The catheter may be used to treat (e.g., break up) lesions at the treatment site by generating shock waves using the at least one shock wave emitter 108 that impinge upon the lesions.

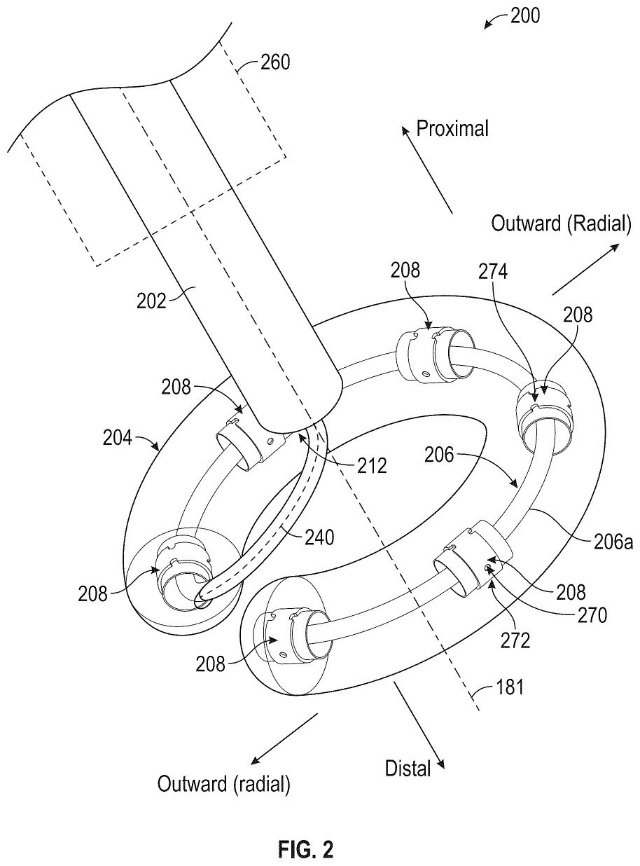

FIG. 2 illustrates aspects of a shock wave catheter 200 that enables a user to position a plurality of shock wave emitters 208 closer to a lesion within a body lumen and having an enclosure 204 formed as a non-occluding balloon (i.e., a balloon having an opening to allow fluid to flow through). Advantageously, the non-occluding balloon allows for blood flow to continue through the center of the balloon while delivering therapy, which may allow treatment times to be extended with less risk of tissue damage. In some embodiments, an additional sheath may be included for delivery of the catheter including a non-occluding balloon.

Catheter 200 may include a catheter body 202 (e.g., an elongate sheath) and an emitter support member 206. The plurality of shock wave emitters may be mounted to a pre-formed distal portion 206a at a distal end portion of the emitter support member 206. The pre-formed distal portion 206a may be positioned at least partially within the enclosure 204 and may enable a user to position the plurality of shock wave emitters 208 closer to a lesion within a body lumen. For instance, the pre-formed distal portion 206a and the enclosure 204 may be configured to coil into an annular loop around a longitudinal axis of the catheter body. The enclosure 204 may be attached at its proximal end to the emitter support member 206. The enclosure 204 may be free-floating at its distal end (e.g., not attached to the emitter support member 206) and may enclose the distal end of the emitter support member 206. A fluid lumen 240 may be configured to fill the enclosure 204 (e.g., with a conductive fluid) and/or withdraw the fluid from enclosure 204 to deflate/collapse the enclosure.

The plurality of shock wave emitters 208 may be positioned on the pre-formed distal portion 206a such that when the pre-formed distal portion 206a is positioned distally of a distal end of the catheter body 202, the plurality of shock wave emitters 208 are spaced circumferentially from one another about a longitudinal axis 281 of catheter 200. In some examples, each of the plurality of shock wave emitters 108 may be formed by a respective emitter sheath positioned adjacent to and spaced apart from a conductor (e.g., a conductive wire) by a spark gap (e.g., as illustrated in FIG. 1B and/or as illustrated in FIGS. 3E-7, 10A-12C), thus forming an electrode pair. In some examples, one or more of the emitter sheaths may form an electrode of a plurality of shock wave emitters, and a plurality of emitter sheaths may be positioned on the pre-formed distal portion. In some examples, the plurality of shock wave emitters 208 may be positioned at the same longitudinal location as one another but circumferentially offset from one another when the emitter support member 206 is deployed from the distal opening 212, thus forming a “halo” array, as illustrated in FIG. 1.

In some examples, at least some of the electrode pairs of the plurality of shock wave emitters 208 may be positioned on a circumferential location of the emitter support member 206 that faces radially outward (e.g., 180 degrees from the side of emitter support member 106 that faces longitudinal axis 181). Thus, shock waves generated using the at least one shock wave emitter 208 may be directed outward away from the longitudinal axis 281. In some examples, at least some of the electrode pairs of the plurality of shock wave emitters 208 may be positioned between 0 degrees and 180 degrees offset from the circumferential location of emitter support member 206 that faces longitudinal axis 281. For instance, at least some of the electrode pairs of the plurality of shock wave emitters 208 may be positioned at 90 degrees offset from the circumferential location of emitter support member 206 that faces longitudinal axis 281. Thus, shock waves generated using the at least one shock wave emitter 208 may be directed distally (e.g., when positioned on distal-facing circumferential location 272 of the emitter support member 206) or proximally (e.g., when positioned on proximal-facing circumferential location 274 of the emitter support member 206).

The emitter support member 206 may be configured such that it can be translated longitudinally relative to the catheter body 102 such that it can be retracted into (and deployed from) the catheter body 202. The pre-formed distal portion 206a portion may be straightened as it is pulled into a distal opening 212 of the catheter body 202 and the pre-formed distal portion 206a may naturally coil into the annular loop or “halo” shape as shown in FIG. 2 when deployed from the catheter body 202 (e.g., extended out the distal opening 212). The pre-formed distal portion 206a may be or include a pre-formed nitinol wire or other shape memory material. In some examples, the enclosure 204 is configured such that it can be retracted into the catheter body 202. The enclosure 204 may be pulled proximally in tandem with the emitter support member 206 and may be drawn into the distal opening 212 of catheter body 202. In some examples, the emitter support member 206 is fixedly attached to the catheter body and the enclosure is sealed to the emitter support member 206 such that the emitter support member 206 and enclosure 206 do not slide/translate relative to the catheter body 202. In some examples, the emitter support member 206 is fixedly attached to the catheter body 202 and the catheter body 202, emitter support member 206, and enclosure 204 are retractable into and deployable from an outer sheath 260 (e.g., an introducer).

A radial 360-degree emitter catheter (such as those shown in FIGS. 1 and 2) allows for effective lithotripsy treatment of larger circular structures such as calcified structural heart valves like the aortic and mitral valves by positioning emitters closer to the calcifications and effectively increasing the sonic energy delivered to the target tissue.

In some embodiments, shock wave emitters are directionally biased to emit shock waves that propagate in a radially outward direction from a central axis of the catheter. For example, an emitter band of a shock wave emitter may include a conductive surface provided on a hole or a slot (as further described below) that faces radially outward from a central axis of the catheter. In other words, directional emitters lithotripsy treatment can be focused radially outward for a more targeted and efficient therapy. As discussed with reference to FIGS. 1 and 2, one or more shock wave emitters may additionally, or alternatively, be directionally biased in a distal or proximal direction, which may enable focused shockwave treatment, for instance, applied to valve leaflets.

In some embodiments, deployment of a catheter may include mounting emitters on a pre-formed nitinol wire that is pulled into a straight configuration within a sheath. Once in position a sliding mechanism (e.g., one that is connected to a switch at a proximal catheter handle) may deploy the pre-formed nitinol wire with emitters, allowing for a larger radial coverage of 10 mm to 40 mm while being able to be delivered in a small profile under 18F (less than 6 mm in diameter).

Another approach to increasing sonic energy delivered to target tissue is to reduce destructive interference of the sonic energy by structural features of the catheter. The catheters shown in FIGS. 1 and 2 may include emitter bands having a hole. A conductive surface of the hole forms at least a part of an electrode of an electrode pair. The other electrode of the electrode pair may be formed by a conductive member (e.g., an uninsulated region of a wire) that is positioned radially inward of the emitter band and separated from the emitter band by an insulating member. While this structure may be effective for some anatomies, the emitter band itself may impede propagation of sonic energy generated at the hole.

FIGS. 3A-3D illustrate various emitter bands having slots, instead of holes, that may form a part of a shock wave emitter in a shock wave catheter, according to aspects of the disclosure. FIG. 3A illustrates an emitter band 300A that includes a slot that extends 60 degrees around a circumference of the band. FIG. 3B illustrates an emitter band 300B that includes a slot that extends 90 degrees around a circumference of the band. FIG. 3C illustrates an emitter band 300C that includes a slot that extends 120 degrees around a circumference of the band. FIG. 3D illustrates an emitter band 300D that includes two slots that each extends 120 degrees around a circumference of the band. In some embodiments, an emitter band has one or more slots that extend one to 359 degrees around a circumference of the emitter band. By including slots to the emitter band instead of holes, energy is able to more efficiently be released, minimizing destructive interference and increasing sonic output. Including slots in emitter bands may also help to increase the flexibility and navigability of the distal region of the catheter. FIGS. 3E and 3F illustrate inner electrode configurations that may be used for any of emitter bands 300A-300D. FIG. 3E illustrates an example inner electrode configuration that forms two shock wave emitters (labeled emitter 1 and emitter 2) together with emitter band 302. Emitter band 302 may include any of the aspects described with reference to emitter bands 300A-300D. Emitter band 302 may include an elongate slot 301 extending at least partially around a circumference of emitter band 302. The slot 301 may extend between approximately 30 degrees and 359 degrees around the circumference.

A first inner electrode 308 may be positioned radially inward of emitter band 302 and aligned with a first portion of the slot 301. The first inner electrode 308 may be spaced apart from an edge of the slot 301 by a spark gap. A second inner electrode 304 may be positioned radially inward of emitter band 302 and aligned with a second portion of the slot 301. The first inner electrode 308 and second electrode 304 may be spaced apart from one another by a distance d (which may be measured from a center or centroid of the first inner electrode 308 and second inner electrode 304. In some examples, the distance d may be optimized to promote constructive interference of shock waves generated using the first and second inner electrodes. In some examples, the distance d is between 4.0 mm and 5.0 mm. In some examples, the distance d is between 1.0 mm and 7.0 mm.

In some examples, the first inner electrode 308 is formed by or connected to a conductive portion of a wire 310 and/or the second inner electrode 304 is formed by or connected to a conductive portion of another wire 306. Wire 310 may be electrically connected to a first (e.g., positive or negative) terminal of an energy source and wire 306 may be connected to a second terminal (e.g., positive or negative, opposite of the first terminal). When a voltage is applied across the first and second terminal, an electrical current may arc across the spark gap formed between inner electrode 308 and the edge of slot 301 generating a first shock wave and an electrical current may arc across the spark gap formed between inner electrode 304 and the edge of slot 301 generating a second shock wave.

In some examples, the first inner electrode 308 and/or second inner electrode 304 may be a conductive sheath, flattened wire end, conductive cylinder connected to a conductive portion of a wire, or other conductive member. The first inner electrode 308, second inner electrode 304, and/or emitter band 302 may be formed from any conductive material, for instance, including copper, stainless steel, tungsten, molybdenum, clad wire, etc. In some examples, an additional one or more inner electrodes may be spaced apart from a second elongate slot formed into a different portion of emitter band 302 (e.g., as depicted in FIG. 3D). The additional one or more inner electrodes may form an additional one or more shock wave emitters together with emitter band 302.

FIG. 3F illustrates an example inner electrode configuration that forms one shock wave emitter together with an emitter band 312. In some examples, the example depicted in FIG. 3F may form one of a plurality of shock wave emitters on a catheter wired according to the example discussed below with reference to FIG. 5 (where the inner electrode is the cathode for each emitter and the emitter band is the anode for each emitter) or FIG. 7 (where the inner electrode is the anode for each emitter and the emitter band is the cathode for each emitter). Emitter band 312 may include any of the aspects described with reference to emitter bands 300A-300F. Emitter band 312 may include an elongate slot 311 extending at least partially around a circumference of emitter band 312. The slot 311 may extend between approximately 10 degrees and 359 degrees around the circumference (e.g., 30 degrees, 60 degrees, 90 degrees, 120 degrees, 160 degrees, etc.). An inner electrode 314 may be positioned radially inward of emitter band 312 and aligned with the slot 311. The inner electrode 314 may be spaced apart from an edge of the slot 311 by a spark gap. In some examples, inner electrode 314 is formed by or connected to a conductive portion of a wire 316. Wire 316 may be electrically connected to a first (e.g., positive or negative) terminal of an energy source. In some examples, another wire 318 may be electrically connected (e.g., soldered, welded, adhered, etc.) to the emitter band 312. Wire 318 may be connected to a second terminal (e.g., positive or negative, opposite of the first terminal). When a voltage is applied across the first and second terminal, an electrical current may arc across the spark gap formed between inner electrode 308 and the edge of slot 301 generating one or more shock waves. In some examples, the inner electrode 316 may be a conductive sheath, flattened wire end, conductive cylinder connected to a conductive portion of a wire, or other conductive member. The inner electrode 316, wire 318, and/or emitter band 312 may be formed from any conductive material, for instance, including copper, stainless steel, tungsten, molybdenum, clad wire, etc.

FIG. 3G illustrates a graph showing the relative improvement in sonic output emitter bands having a conventional configuration with two holes spaced 180 degrees apart, 90 degrees apart, and 60 degrees apart, respectively, around the circumference of the band compared to slotted emitter bands (e.g., as shown in FIGS. 3A-3F). The 120-degree slotted emitter band provided the most sonic output overall, followed by the 90-degree slotted emitter band and the 60-degree slotted emitter band, respectively. The conventional emitter bands with two holes spaced 180 degrees, 90 degrees, and 60 degrees apart, respectively, provided lower sonic output than each of the slotted emitter bands.

Another improvement for shock wave catheters disclosed herein improves their durability and longevity. Durability of lithotripsy catheters using an electrohydraulic mechanism of action (such as those described above) may be decreased after the repeated arcing at the electrode pairs required to generate shock waves and treat target tissue. In particular, arcing and shock wave generation may degrade more of the anode than the cathode due to the high impact forces experienced by the anode. In some examples, the shock wave emitters disclosed herein are configured such that a larger or otherwise more durable electrode serves as the anode. For instance, by directing the arc so that the emitter band (such as those having slots or holes described above) is always the anode (i.e., the electrode receiving the current arc), durability and efficiency of shock wave catheters can be greatly increased. In some embodiments, a shock wave catheter may be configured such that each electrode of the electrode pairs having a larger surface area is the anode. In some embodiments, a shock wave catheter may be configured such that each electrode of the electrode pairs having a higher durability is the anode. Durability may be characterized by the electrode material hardness or melting temperature. In some embodiments, durability may be inverse to the thermal conductivity of the electrode material.

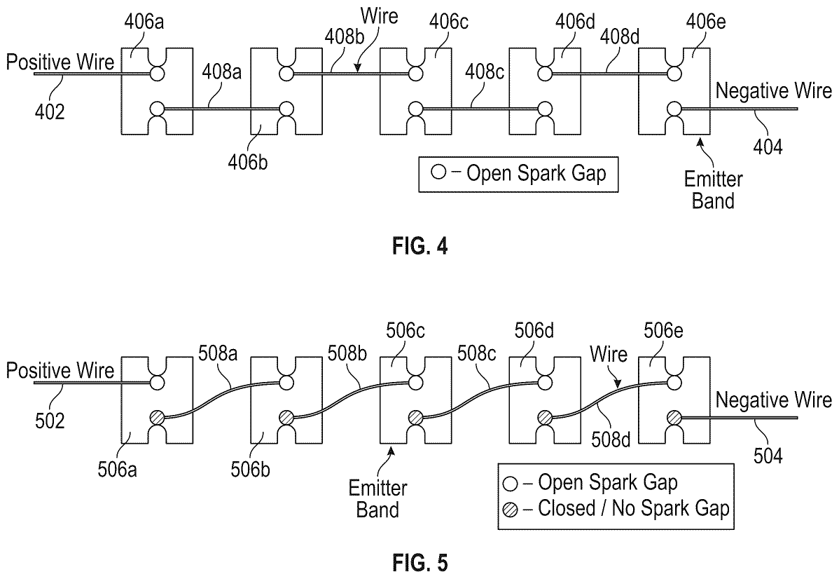

FIG. 4 illustrates the wiring of a known shock wave catheter, such as those described in U.S. Pat. No. 8,888,788. The known catheter is configured such that each emitter band includes two electrode pairs, where the emitter band acts as a cathode in one electrode pair and an anode in the other electrode pair. In the series connection shown in FIG. 4, each wire at one end is an anode and at the other end is a cathode. The wiring diagram in FIG. 4 shows a plurality of emitter bands 406a-406e. A positive wire 402 carries an electrical current from a power source to a first emitter band 406a. The positive wire is positioned adjacent to the first emitter band 406a but spaced apart from the first emitter band 406a by a first spark gap. A first portion of a second wire 408a is positioned adjacent to the first emitter band 406a and spaced apart from the first emitter band 406a by a another spark gap. The second wire 408a extends to a second emitter band 406b and a second portion of the second wire 408a is positioned adjacent to and spaced apart from the second emitter band 406b by another spark gap. Additional wires 408b, 408c, and 408d are daisy chained in this manner between emitter bands 406b, 406c, 406d, and 406e. Each wire 408a-408d may include a first end and a second end. A first end of each respective wire may be positioned adjacent to and spaced apart from one of the emitter bands by a spark gap, and a second end of each respective wire may be positioned adjacent to and spaced apart from another one of the emitter bands by another spark gap. A negative wire 404 may be positioned adjacent to and spaced apart from the last emitter band in the chain (e.g., emitter band 406e in FIG. 4) by a spark gap. Thus, when a voltage is applied between the positive wire and the negative wire, an electrical current will jump from the positive wire to the first emitter band 406a across a spark gap, resulting in a shock wave, and then jump from the first emitter band 406a to the first wire 408a, resulting in another shock wave. The current will likewise jump from the first wire 408a to the second emitter band 408b, resulting in a shock wave, and from emitter band 406b to wire 408b, resulting in another shock wave. Thus, as noted above, each emitter band (406a-406e) acts as a cathode in one electrode pair and an anode in the other electrode pair.

FIG. 5 illustrates directional wiring of a shock wave catheter, according to aspects of the disclosure. In this embodiment, each emitter band includes a single electrode pair such that each wire acts only as the cathode in each electrode pair. Thus, the electrical current always jumps from a respective wire to a respective emitter band and not from the emitter band to the respective wire. The emitter bands include holes or slots as described above. In some embodiments, the emitter bands include a conductive edge (e.g., a proximal or distal edge) that serves as an anode for receiving the arcing.

The wiring diagram in FIG. 5 shows a plurality of emitter bands 506a-506e. Similar to the wiring diagram shown in FIG. 4, a positive wire 502 carries an electrical current from a power source to a first emitter band 506a. The positive wire 502 is positioned adjacent to an emitter band 506a but spaced apart from the emitter band 506a by a first spark gap. A first portion of another wire 508a is electrically connected to the emitter band 506a. The first portion of the wire 508a may be soldered, welded, clamped, or otherwise electrically connected to the emitter band 506a. Wire 508a extends to another emitter band 506b and a second portion of the wire 508a is positioned adjacent to and spaced apart from the emitter band 506b by another spark gap. A first end of another wire 508b is electrically connected (e.g., soldered, welded, clamped, etc.) to emitter band 506b and wire 508b extends from emitter band 506b to another emitter band 506c. A second end of wire 508b is positioned adjacent to and spaced apart from emitter band 506c by another spark gap. A first end of another wire 508c is electrically connected to emitter band 506c. Wire 508c extends to another emitter band 506d and a second end of wire 508c is positioned adjacent to and spaced apart from emitter band 506d by another spark gap. A first end of another wire 508d is electrically connected to emitter band 506d. Wire 508d extends to another emitter band 508e and a second end of wire 508d is positioned adjacent to and spaced apart from emitter band 508d. Finally, a negative wire 504 is electrically connected to emitter band 508d on a first end, and a second end of negative wire 504 is connected to a negative terminal of the voltage source. When a voltage is applied across positive wire 502 and negative wire 504, an electrical current jumps across each of the respective spark gaps from a respective one of the wires to a respective one of the emitter bands. Unlike the wiring diagram shown in FIG. 4, the wiring diagram shown in FIG. 5 is configured such that the emitter bands always serve as the anode of each electrode pair. As discussed above, this configuration results in relatively lower stresses on the respective wires forming electrodes of respective electrode pairs because the anode (in this example, the emitter bands) experiences a relatively higher amount of stress during shock wave generation.

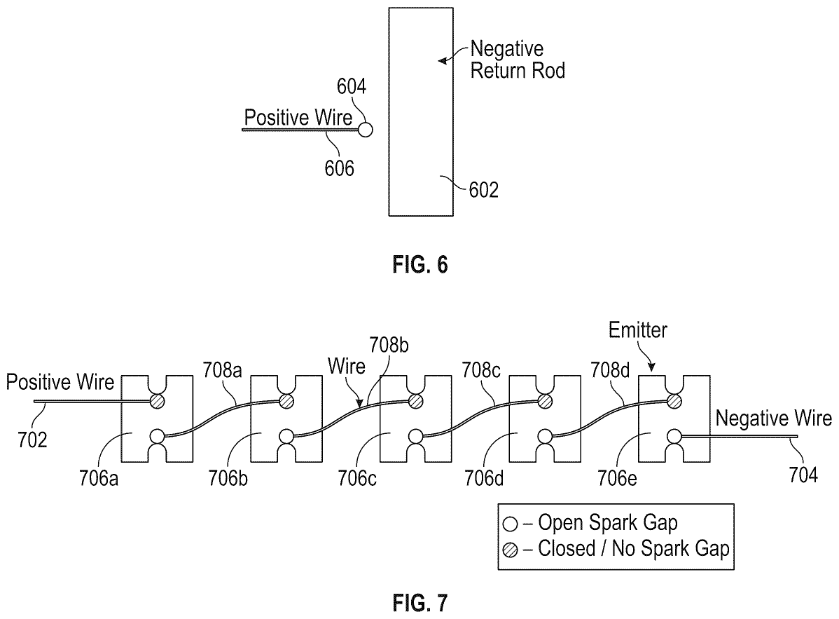

FIG. 6 illustrates directional arcing of a shock wave catheter, according to aspects of the disclosure, where the anode is an alternative shape from a band. As shown in the drawing, the anode may be a rod 602 and a cathode 604 may be a conductive end of a wire 606. In some embodiments, the anode is another more durable or thicker wire or other conductive element. For instance, according to some examples the cathode may be a copper conductive member and the anode may be formed from a more durable material such as molybdenum, tungsten, rhodium, rhenium, tantalum, niobium, or a combination thereof.

FIG. 7 illustrates directional arcing of a shock wave catheter, according to aspects of the disclosure, where the anode is a wire end and the cathode is an emitter band. While such a configuration may be less durable for the reasons described above, it may generate higher sonic output and may be useful for treating lesions where higher peak sonic output is desired over repeated pulses of relatively lower peak power. The configuration illustrated in FIG. 7 may produce higher sonic output because the anode is formed by a discrete focal point and configuring the shock wave emitter such that the current jumps to a discrete focal point such as a wire end may produce higher sonic output. As illustrated in FIG. 7, in each electrode pair, a wire end forms the anode, and a portion of an emitter band forms the cathode in each electrode pair. The wiring diagram in FIG. 7 shows a plurality of emitter bands 706a-706e. Similar to the wiring diagram shown in FIG. 4, a positive wire 702 carries an electrical current from a power source to a first emitter band 706a. However, in contrast to the configuration of FIG. 5, here the positive wire 702 is electrically connected to the emitter band 706a. A first portion of another wire 708a is positioned adjacent to and spaced apart from emitter band 706a by a spark gap.

Wire 708a extends to another emitter band 706b and a second portion of the wire 708a is electrically connected to emitter band 706b.

A first end of another wire 708b is positioned adjacent to and spaced apart from emitter band 706b and wire 708b extends from emitter band 706b to another emitter band 706c. A second end of wire 708b is electrically connected to emitter band 706c. A first end of another wire 708c is positioned adjacent to and spaced apart from emitter band 706c. Wire 708c extends to another emitter band 706d and a second end of wire 708c is electrically connected to emitter band 706d. A first end of another wire 708d is is positioned adjacent to and spaced apart from emitter band 706d. Wire 708d extends to another emitter band 708e and a second end of wire 708d electrically connected to emitter band 708d.

Finally, a negative wire 704 is positioned adjacent to and spaced apart from emitter band emitter band 708d on a first end, and a second end of negative wire 704 is connected to a negative terminal of the voltage source. When a voltage is applied across positive wire 702 and negative wire 704, an electrical current jumps across each of the respective spark gaps from a respective one of the emitter bands to a respective one of the wires. Unlike the wiring diagram shown in FIG. 5, the wiring diagram shown in FIG. 5 is configured such that the emitter bands always serve as the cathode of each electrode pair and the wire ends always serve as the anode of the respective electrode pair. As discussed above, this configuration results in relatively higher stresses on the respective wires but may provide higher sonic output.

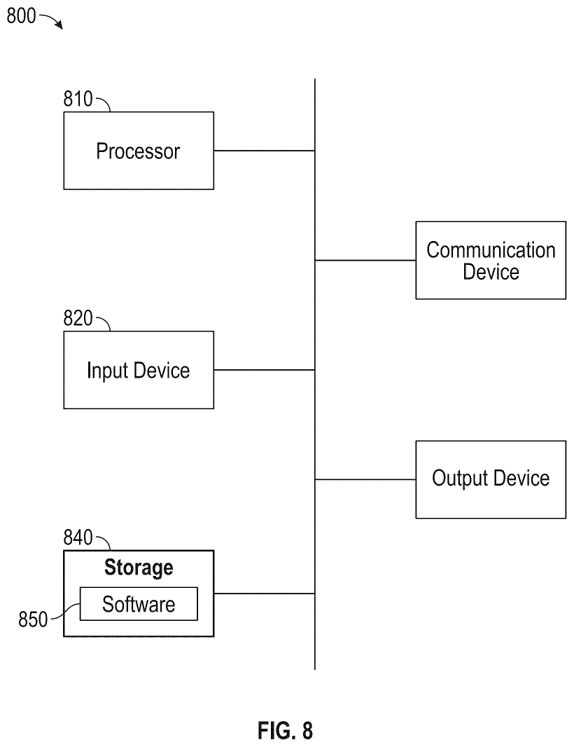

FIG. 8 illustrates an example of a computing system 800 that may be used for controlling shock wave generation from the catheters described herein. System 800 can be a computer connected to a network, such as one or more networks of hospital, including a local area network within a room of a medical facility and a network linking different portions of the medical facility, or a wide-area network accessed through the internet or other means. System 800 can be a client or a server. System 800 can be any suitable type of processor-based system, such as a personal computer, workstation, server, handheld computing device (portable electronic device), such as a phone or tablet, or dedicated device. System 800 can include, for example, one or more of input device 820, output device 830, one or more processors 810, storage 840, and communication device 860. Input device 820 and output device 830 can generally correspond to those described above and can either be connectable or integrated with the computer.

Input device 820 can be any suitable device that provides input, such as a touch screen, keyboard or keypad, mouse, gesture recognition component of a virtual/augmented reality system, or voice-recognition device. Output device 830 can be or include any suitable device that provides output, such as a display, touch screen, haptics device, virtual/augmented reality display, or speaker.

Storage 840 can be any suitable device that provides storage, such as an electrical, magnetic, or optical memory including a RAM, cache, hard drive, removable storage disk, or other non-transitory computer-readable medium. Communication device 860 can include any suitable device capable of transmitting and receiving signals over a network, such as a network interface chip or device. The components of the computing system 800 can be connected in any suitable manner, such as via a physical bus or wirelessly.

Processor(s) 810 can be any suitable processor or combination of processors, including any of, or any combination of, a central processing unit (CPU), field programmable gate array (FPGA), and application-specific integrated circuit (ASIC). Software 850, which can be stored in storage 840 and executed by one or more processors 810, can include, for example, the programming that embodies the functionality or portions of the functionality of the present disclosure (e.g., as embodied in the devices as described above), such as programming for performing one or more steps of method 200, method 300, and/or method 600.

Software 850 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 840, that can contain or store programming for use by or in connection with an instruction execution system, apparatus, or device.

Software 850 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport computer-readable medium can include, but is not limited to, an electronic, magnetic, optical, electromagnetic, or infrared wired or wireless propagation medium.

System 800 may include a sensor device 870 that provides sensor data for processing by processor 810. Sensor device 870, in some embodiments, may be an imaging sensor that provides imaging data, for a lesion being treated. In some embodiments, sensor device 870 may be a voltage sensor, a current sensor, a pressure sensor, a temperature sensor, or an optical sensor for providing data about a state of the catheter or a lesion.

System 800 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 800 can implement any operating system suitable for operating on the network. Software 850 can be written in any suitable programming language, such as C, C++, Java, or Python. In various examples, application software embodying the functionality of the present disclosure can be deployed in different configurations, such as in a client/server arrangement or through a Web browser as a Web-based application or Web service.

System 800 may be configured to selectively control the delivery of energy from one or more of energy sources (e.g., a voltage pulse generator or a light energy source) to one or more acoustic energy emitters (e.g., a forward-firing emitter, a radially-firing emitter, an unenclosed emitter, or an enclosed emitter) depending on input from input device 820.

System 800 may be configured to tune the energy properties of energy delivered to one or more of the above-described emitters based on tissue properties received from sensor device 870. Tissue properties may include lesion tissue type (e.g., calcific, thrombic, fibrotic), lesion morphology (e.g., thickness, length, eccentricity).

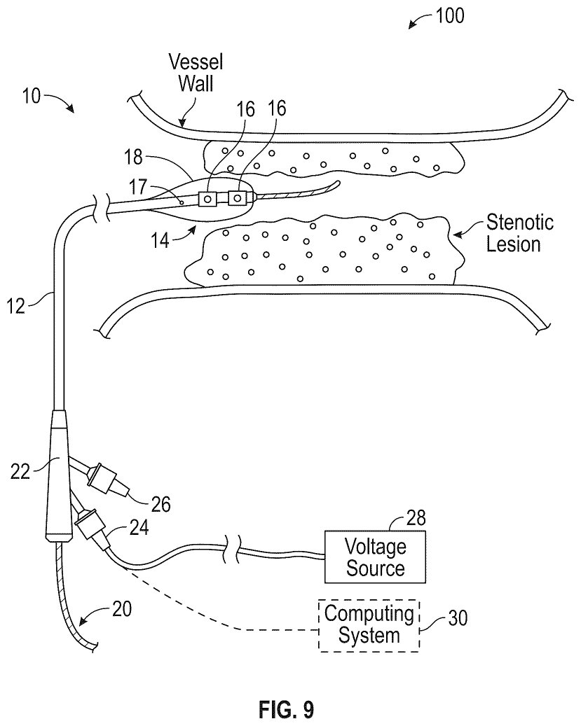

FIG. 9 illustrates a system 900 for treating hardened lesions in a body of a subject. System 900 may include any or all of the features described with reference to FIGS. 1-8. The system 900 includes a catheter 10 for generating shock waves. The catheter 10 generates shock waves that fragment, crack, or otherwise break up hardened lesions within the body. For example, the catheter 10 can be used to treat calcifications within blood vessels, the heart (e.g., valves), kidneys, bladder, and gallbladder. The catheter 10 includes a plurality of shock wave emitters 16 positioned within an enclosure 18. The catheter 10 is advanced to a lesion in a patient's body, such as the stenotic lesion depicted in FIG. 1, over a guidewire 20 carried in a guidewire sheath. Voltage pulses are applied to the shock wave emitters 16 to generate shock waves. The shock wave emitters 16 each include electrode pairs having first and second electrodes separated by a gap (a “spark gap”). When a voltage pulse is applied to a shock wave emitter 16, current flows across the gap between the electrodes of the shock wave emitter 16.

An enclosure 18 is attached to the distal end 14 of the catheter 10, forming an annular channel around a body 12 of the catheter 10. The enclosure 18 surrounds the plurality of shock wave emitters 16. The enclosure 18 is fillable with a conductive fluid, such as saline. The enclosure 18 can be compliant (e.g., a low-profile flexible angioplasty balloon, a polymer membrane in tension that can flex outward, etc.) such that it expands when filled or may be noncompliant such that it maintains a substantially constant volume and profile when filled. The conductive fluid enables current to flow across the gaps between electrodes of the shock wave emitters to generate shock waves. The shock waves propagate within the conductive fluid outwardly from the electrode pairs of the shock wave emitters 16 through the walls of the enclosure 18 and then into the target lesion. In one or more examples, the conductive fluid contains x-ray contrast fluid for fluoroscopic viewing of the catheter 10 during use. The enclosure 18 may mitigate thermal injury to soft tissue and reduce cavitation stresses by limiting expansion of the vapor bubbles produced during shock wave generation. For instance, the vapor bubbles hit the enclosure wall before reaching their maximum potential size, thus inducing collapse, and reducing cavitation stress and preventing soft tissue injury that can be caused by tensile stresses during cavitation bubble collapse.

The catheter 10 includes a proximal end 22 (which may include or form a handle) that remains outside of a patient's vasculature during treatment. The proximal end 22 includes an entry port for receiving the guidewire 20. The proximal end 22 includes at least one fluid port 26 for filling and emptying the enclosure 18 during treatment. An electrical connection port 24 is also located on the proximal end 22 to provide an electrical connection between the distal shock wave emitters 16 and an external voltage source 28.

The catheter body 12 extends from the proximal end 22 to the distal end 14 of the catheter 10. The catheter body 12 provides various internal conduits connecting elements of the distal end 14 with the proximal end 22 of the catheter. The catheter body 12 includes an elongate tube that includes a lumen for receiving the guidewire 20. The elongate tube may include additional lumens extending through the catheter body 12 or along an outer surface of the catheter body 12. For example, fluid lumens (e.g., a fluid inlet lumen and a fluid outlet lumen or a combined flush lumen) can be located along or within the catheter body 12 for carrying conductive fluid from the fluid port 26 into the enclosure 18.

In some examples, one or more sensors 17 are positioned along the catheter 10. The sensors 17 may be positioned at any location on catheter 10. For instance, the sensors 17 may be positioned proximal to one or more shock wave emitters 16, distal from one or more shock wave emitters 16, and/or intermediary between one or more shock wave emitters 16 (or any combination thereof). The sensors 17 include one or more of any suitable sensor devices, such as a pressure sensor, a thermal sensor, an electrical sensor (e.g., current, voltage, resistance, and/or impedance sensors), or a visualization element. Measurements from sensors 17 can be used to provide feedback to an operator who is providing treatment using catheter 10. For example, feedback based on measurements from sensor 17 can be generated and displayed to an operator via a display of a computing system 30. The feedback may provide information indicating a status of the catheter 10, such as the temperature and/or pressure within the enclosure 18 and/or the voltage and/or current supplied to the shock wave emitters 16. The feedback provided via the computing system 30 may provide guidance on further steps for the operator to implement with catheter 10. For example, in implementations where sensor 17 includes a pressure sensor, a slight decrease in pressure that is displayed via the display of the computing system 30 may indicate success at cracking a calcified lesion, due to the fact that the expandable member surrounding the emitters is able to further expand without changing the volume of fluid within the expandable member, guiding the user to move the catheter 10 to treat another region of a lesion. Further, a significant decrease in pressure may indicate a rupture failure mode where the expandable member has lost seal and fluid volume, and thus guiding toward withdrawal of the device. In implementations where the sensor 17 includes a visualization element, an operator of the catheter 10 may be able to more clearly understand where the catheter device 10 is located relative to a target lesion or anatomy, prior to, during, and after delivering shock wave treatment to a lesion.

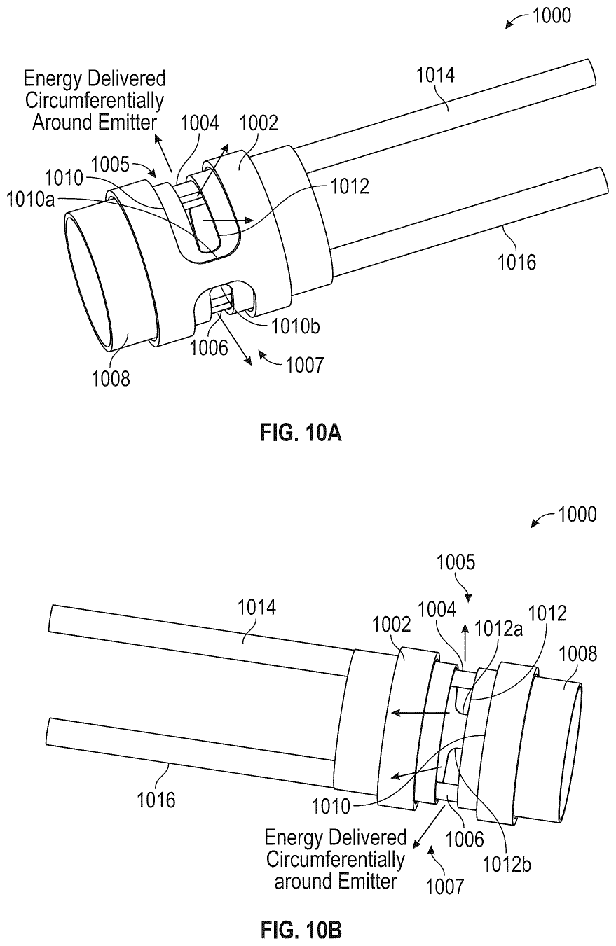

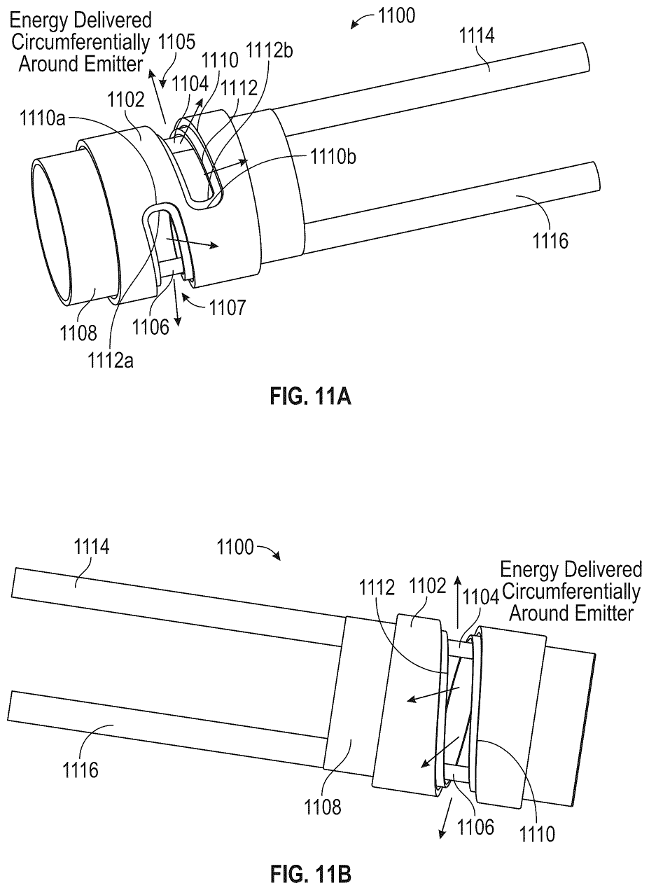

As discussed with reference to FIGS. 3A-3D, emitter bands having circumferentially extending slots may enable increased uniformity in sonic output circumferentially around the emitter band. FIGS. 10A-11B illustrate examples of shock wave emitter assemblies that include emitter bands having circumferentially extending slots.

With reference to FIGS. 10A-10B, an exemplary shock wave emitter assembly 1000 includes a conductive band 1002 that has one or more circumferentially extending elongate slots 1010. The shock wave emitter assembly 1000 may be used to form the shock wave emitters of any of the catheters disclosed herein, including the shock wave emitters of the catheters illustrated in FIGS. 1-2, and the shock wave emitters 16 of catheter 10 of FIG. 9. FIG. 10A illustrates a first perspective of shock wave emitter assembly 1000 and FIG. 10B illustrates a second perspective of shock wave emitter assembly 1000. The at least one elongate slot 1010 may be formed in conductive band 1002 and may extend circumferentially around the conductive band 1002 from a first end 1010a to a second end 1010b of the slot 1010. An edge of the conductive band 1002 defining the elongate slot 1010 may form an outer electrode of one or more shock wave emitters of shock wave emitter assembly 1000. One or more inner electrodes, for instance, inner electrode 1004 and inner electrode 1006 may be positioned radially inward of the conductive band 1002 and may form respective inner electrodes of the one or more shock wave emitters. Thus, inner electrode 1004 and a portion of the edge of conductive band 1002 defining elongate slot 1010 together may form a first shock wave emitter 1005 and inner electrode 1006 and a portion of the edge of conductive band 1002 defining elongate slot 1010 together may form a second shock wave emitter 1007.

Inner electrode 1004 and/or inner electrode 1006 may be aligned with the elongate slot 1010 radially inward of the emitter band 1002. In some examples, an insulating layer 1008 is positioned between the conductive band 1002 and the one or more inner electrodes (e.g., inner electrode 1004 and inner electrode 1006). At least one elongate slot 1012 may be formed in the insulating layer and may extend circumferentially around the circumference of the insulating layer between end 1012a and 1012b. The elongate slot 1012 may be aligned with the elongate slot 1010 formed in conductive band 1002. The insulating layer may prevent contact between the one or more inner electrodes (e.g., 1004 and 1006) and the conductive band 1002.

The inner electrodes 1004 and 1006 are thus spaced apart from the conductive band 1002 and positioned adjacent to an edge of the elongate slot 1010. Inner electrode 1004 and inner electrode 1006 may be formed by removing a portion of an insulating sleeve from insulated wire 1014 and insulated wire 1016, respectively. Thus, inner electrode 1004 and inner electrode 1006 may each be or include a conductive portion of insulated wire 1014 and insulated wire 1016, respectively. In some examples, one or both of inner electrode 1004 and inner electrode 1006 include one or more additional conductive members, such as a semi-cylindrical conductive member, electrically connected to the uninsulated conductive portion of insulated wire 1014 and insulated wire 1016, respectively. Wires 1014 and 1016 may be respectively connected to a supply and return (e.g., positive and negative) terminal of a voltage source. When a voltage is applied across wires 1014 and 1016, one or more shock waves may be generated using shock wave emitter 1005 and shock wave emitter 1007. For instance, when the voltage is applied across wires 1014 and 1016, an electrical current jumps across the gap between inner electrode 1004 and conductive band 1002 and between the gap formed between inner electrode 1006 and conductive band 1002, resulting in one or more shock waves. The shock waves propagate radially outward from conductive band 1002. The sonic output of the one or more shock waves may be distributed circumferentially around the elongate slot 1010. In some examples, elongate slot 1010 promotes a relatively more uniform distribution of sonic output, for instance, compared to conductive bands having holes in place of elongate slots.

The conductive band 1002 may include or be formed from stainless steel and the insulating layer 1008 may include or be formed from polyimide. The conductive band 1002 may be attached to the insulating layer 1008 using an adhesive. While the conductive band 1002 and insulating layer 1008 are illustrated in FIGS. 10A and 10B as having a single continuous elongate slot extending between ends 1010a and 1010b and between 1012a and 1012b, respectively, it should be understood that conductive band 1002 and insulating layer 1008 may include a plurality of discontinuous elongate slots. The plurality of discontinuous elongate slots may be longitudinally aligned with one another (e.g., as illustrated in FIG. 3D) and/or longitudinally offset from one another.

In some examples the elongate slot 1010 may extend up to 120 degrees, up to 130 degrees, up to 140 degrees, up to 150 degrees, up to 160 degrees, up to 170 degrees, up to 180 degrees, up to 190 degrees, up to 200 degrees, up to 210 degrees, up to 220 degrees, up to 230 degrees, up to 240 degrees, up to 250 degrees, up to 260 degrees, up to 270 degrees, up to 280 degrees, up to 290 degrees, up to 300 degrees, up to 310 degrees, up to 320 degrees, up to 330 degrees, up to 340 degrees, up to 350 degrees, and/or up to 359 degrees around the circumference of the conductive band 1002. In some examples the elongate slot 1010 may extend at least 120 degrees, at least 130 degrees, at least 140 degrees, at least 150 degrees, at least 160 degrees, at least 170 degrees, at least 180 degrees, at least 190 degrees, at least 200 degrees, at least 210 degrees, at least 220 degrees, at least 230 degrees, at least 240 degrees, at least 250 degrees, at least 260 degrees, at least 270 degrees, at least 280 degrees, at least 290 degrees, at least 300 degrees, at least 310 degrees, at least 320 degrees, at least 330 degrees, at least 340 degrees, at least 350 degrees, and/or at least 359 degrees around the circumference of the conductive band 1002.

In some examples the elongate slot 1012 may extend up to 120 degrees, up to 130 degrees, up to 140 degrees, up to 150 degrees, up to 160 degrees, up to 170 degrees, up to 180 degrees, up to 190 degrees, up to 200 degrees, up to 210 degrees, up to 220 degrees, up to 230 degrees, up to 240 degrees, up to 250 degrees, up to 260 degrees, up to 270 degrees, up to 280 degrees, up to 290 degrees, up to 300 degrees, up to 310 degrees, up to 320 degrees, up to 330 degrees, up to 340 degrees, up to 350 degrees, and/or up to 359 degrees around the circumference of the insulating layer 1008. In some examples the elongate slot 1012 may extend at least 120 degrees, at least 130 degrees, at least 140 degrees, at least 150 degrees, at least 160 degrees, at least 170 degrees, at least 180 degrees, at least 190 degrees, at least 200 degrees, at least 210 degrees, at least 220 degrees, at least 230 degrees, at least 240 degrees, at least 250 degrees, at least 260 degrees, at least 270 degrees, at least 280 degrees, at least 290 degrees, at least 300 degrees, at least 310 degrees, at least 320 degrees, at least 330 degrees, at least 340 degrees, at least 350 degrees, and/or at least 359 degrees around the circumference of the insulating layer 1008.

In some examples, a conductive band used for any of the shock wave emitters disclosed herein may include an elongate slot that extends, at least in part, in a longitudinal direction of the conductive band. For instance, an elongate slot may have a helical shape. FIGS. 11A and 11B illustrate aspects of an exemplary shock wave emitter assembly 1100 including a conductive band 1102 having a helically shaped slot 1110. The shock wave emitter assembly 1100 may be used to form the shock wave emitters of any of the catheters disclosed herein, including the shock wave emitters of the catheters illustrated in FIGS. 1-2, and the shock wave emitters 16 of catheter 10 of FIG. 9. FIG. 11A illustrates shock wave emitter assembly 1100 from a first perspective and FIG. 11B illustrates shock wave emitter assembly 1100 from a second perspective.

The helically shaped slot 1110 may be formed in the conductive band 1102 and extend helically around the conductive band 1102. The slot 1110 may extend helically between a first end 1110a and a second end 1110b such that the first end 1110a is longitudinally spaced apart from the second end 1110b along the length of conductive band 1102. The helically shaped slot 1110 may extend for any number of revolutions (including less than 1 revolution, or less than 360 degrees around the circumference of conductive band 1102, or more than 1 revolution) and for any longitudinal length along conductive band 1002. A helically shaped slot, such as slot 1110, may enable distribution of sonic energy output both circumferentially around the conductive band 1102 and longitudinally along the length of the conductive band 1102.

Conductive band 1102 may form an outer electrode of one or more shock wave emitters of shock wave emitter assembly 1100. An edge of conductive band 1102 defined by slot 1110 may form the outer electrode of one or more shock wave emitters. One or more inner electrodes, for instance, inner electrode 1104 and inner electrode 1106 may be positioned radially inward of the conductive band 1102. The inner electrode 1104 and/or inner electrode 1106 may be aligned with a respective portion of the slot 1110 radially inward of the emitter band 1102. The inner electrode 1104 and inner electrode 1106 may be longitudinally spaced apart from one another along conductive band 1002. For instance, inner electrode 1104 may be positioned proximally of inner electrode 1106. In some examples, inner electrode 1104 and inner electrode 1106 are positioned 180 degrees apart from one another around the circumference of conductive band 1102. In some examples, inner electrode 1104 and inner electrode 1106 are positioned less than 180 degrees apart from one another around the circumference of conductive band 1102. Inner electrode 1104 together with a portion of the edge of conductive band 1102 defined by slot 1110 may form a first shock wave emitter 1105. Inner electrode 1106 together with a portion of the edge of conductive band 1102 defined by slot 1110 may form a first shock wave emitter 1107.

Inner electrode 1104 may include or be an uninsulated conductive portion of an insulated wire 1114. Inner electrode 1106 may include or be an uninsulated conductive portion of an insulated wire 1116. Inner electrode 1104 and inner electrode 1106 may be formed by removing a portion of an insulating sleeve from insulated wire 1114 and insulated wire 1116, respectively. In some examples, one or both of inner electrode 1104 and inner electrode 1106 include one or more additional conductive members, such as a conductive sheath, semi-cylindrical conductive member, etc., electrically connected to the uninsulated conductive portion of insulated wire 1114 and insulated wire 1116, respectively. An insulating layer 1108 may be positioned between the conductive band 1102 and the one or more inner electrodes (e.g., inner electrode 1104 and inner electrode 1106). A helically shaped slot 1112 may be formed in the insulating layer and may extend helically around the circumference of the insulating layer 1108 between end 1112a and 1112b. The slot 1112 may be aligned with the slot 1110 formed in conductive band 1102.

When a voltage is applied across wires 1114 and 1116, one or more shock waves may be generated using shock wave emitter 1105 and shock wave emitter 1107. For instance, when the voltage is applied across wires 1114 and 1116, an electrical current jumps across the gap between inner electrode 1104 and conductive band 1102 and between the gap formed between inner electrode 1106 and conductive band 1102, resulting in one or more shock waves. The shock waves propagate radially outward from conductive band 1102. The sonic output of the one or more shock waves may be distributed along the helically shaped slot 1110. In some examples, the helically shaped slot 1110 promotes a uniform distribution of sonic output along the helical slot.

The conductive band 1102 may include or be formed from stainless steel and the insulating layer 1108 may include or be formed from polyimide. The conductive band 1102 may be attached to the insulating layer 1108 using an adhesive. While the conductive band 1102 and insulating layer 1108 are illustrated in FIGS. 11A and 11B as having a single continuous helically shaped slot (1110 and 1112, respectively) extending between ends 1110a and 1110b and between 1112a and 1112 b, respectively, it should be understood that conductive band 1102 and insulating layer 1108 may include a plurality of discontinuous slots. The plurality of discontinuous slots may be longitudinally offset from one another.

In some examples, a relatively uniform distribution of sonic output may be achieved using additional shock wave emitters distributed around the circumference of a conductive band. For instance, a conductive band may include a plurality of holes positioned at a respective plurality of circumferential locations around a conductive band. One or more conductive members may be positioned radially inward of the plurality of holes in the conductive band and separated from the conductive band by a respective spark gap at each respective hole in the conductive band. By distributing the plurality of holes around the circumference of an emitter band, sonic output generated by the shock wave emitters corresponding to each hole location may also be uniformly distributed around the circumference of the conductive band.

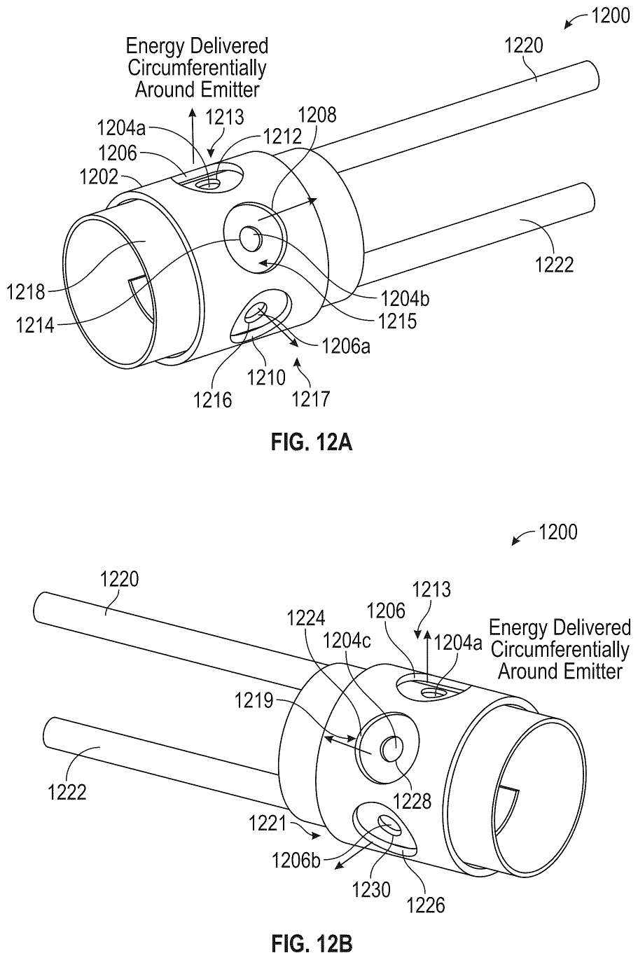

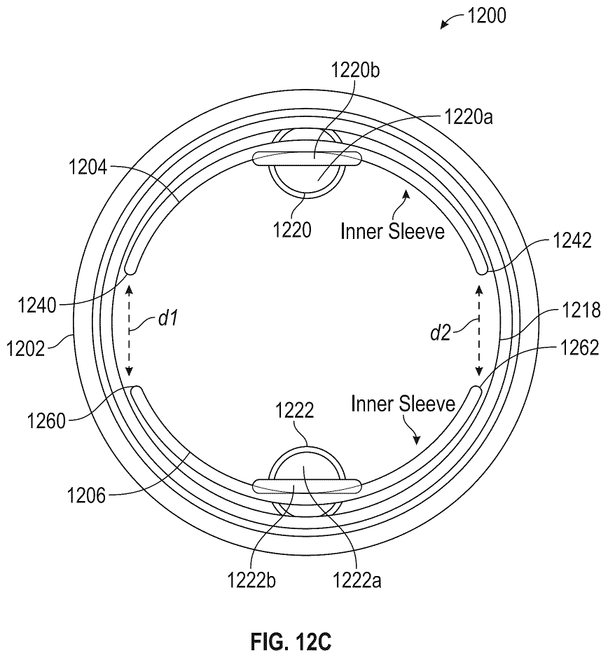

FIGS. 12A and 12B illustrate aspects of an exemplary shock wave emitter assembly 1200 that includes a conductive band 1202 with a plurality of holes, including hole 1206, hole 1208, hole 1210, hole 1224, and hole 1226. FIG. 12A shows shock wave emitter assembly 1200 from a first perspective. FIG. 12B shows shock wave emitter assembly 1200 from a second perspective. The shock wave emitter assembly 1200 may be used to form the shock wave emitters of any of the catheters disclosed herein, including the shock wave emitters of the catheters illustrated in FIGS. 1-2, and the shock wave emitters 16 of catheter 10 of FIG. 9.

An edge defined by each of the plurality of holes, including hole 1206, hole 1208, hole 1210, hole 1224, and hole 1226, may form the outer electrode of a respective shock wave emitter. In some examples, the plurality of holes are uniformly distributed around the circumference of conductive band 1202. In some examples, one or more of the plurality of holes are spaced apart by less than 180 degrees around the circumference of the conductive band from at least two, at least three, or at least four other holes of the plurality of holes around the circumference of the conductive band. In some examples, at least one of the plurality of holes is spaced apart from at least one other of the plurality of holes by 180 degrees around the circumference of the conductive band. In some examples, the plurality of holes are non-uniformly distributed around the circumference of conductive band 1202. In some examples, the plurality of holes are uniformly distributed around the circumference of conductive band 1202. At least two of the plurality of holes may be longitudinally aligned with each other on conductive band 1202. One or more of the plurality of holes may be longitudinally offset on conductive band 1202.

One or more inner electrodes, for instance, inner electrodes 1204a, 1204b, 1204c, and inner electrodes 1206a and 1206b may be positioned radially inward of the conductive band 1202. Each inner electrode may be aligned with a respective one of the plurality of holes. Inner electrode 1204a may be aligned with hole 1206. Inner electrode 1204a and an edge of conductive band 1202 defined by hole 1206 may together form a first shock wave emitter 1213. Inner electrode 1204b may be aligned with hole 1208. Inner electrode 1204b and an edge of conductive band 1202 defined by hole 1208 may together form a shock wave emitter 1215. Inner electrode 1204c may be aligned with hole 1224. Inner electrode 1204c and an edge of conductive band 1202 defined by hole 1224 may together form a shock wave emitter 1219. Inner electrode 1206a may be aligned with hole 1210. Inner electrode 1206a and an edge of conductive band 1202 defined by hole 1210 may together form a shock wave emitter 1217. Inner electrode 1206b may be aligned with hole 1226. Inner electrode 1206b and an edge of conductive band 1202 defined by hole 1226 may together form a shock wave emitter 1221. Another inner electrode may be aligned with another hole positioned 180 degrees around the circumference of conductive band 1202 from hole 1206, forming another shock wave emitter. In some examples, inner electrodes 1204a-1204c may be formed by a first conductor and inner electrodes 1206a-1206b may be formed by a second conductor.

FIG. 12C shows additional detail of the inner electrodes. Inner electrodes 1204a-1204c may be respective portions of a conductive member 1204. Conductive member 1204 may be a semi-cylindrical member having a first end 1240 and a second end 1242. Conductive member 1204 may be electrically connected to a distal end 1220b of a conductive wire 1220a (the conductive portion of insulated wire 1220). Inner electrodes 1206a-1206b may be respective portions of a conductive member 1206. Conductive member 1206 may be a semi-cylindrical member having a first end 1260 and a second end 1262. Conductive member 1206 may be electrically connected to a flattened distal end 1222b of another conductive wire 1222a (the conductive portion of insulated wire 1222). Conductive member 1204 may be spaced apart from conductive member 1206 on a first side by a distance d1 and on a second side by a distance d2. In some examples, d1 and d2 may be equivalent. In some examples, d1 and d2 may be different. In some examples d1 is between 5 degrees and 15 degrees, 5 degrees and 25 degrees, 5 degrees and 35 degrees, or 5 degrees and 45 degrees around the circumference of the conductive band 1202. In some examples d2 is between 5 degrees and 15 degrees, 5 degrees and 25 degrees, 5 degrees and 35 degrees, or 5 degrees and 45 degrees around the circumference of the conductive band 1202. An insulating layer 1218 may be positioned between the conductive band 1202 and the inner electrodes formed by conductive member 1204 and conductive member 1206. The insulating layer 1218 may include a plurality of holes aligned with the plurality of holes formed in the conductive band 1202. A first hole 1212 may be aligned with hole 1206 in the conductive band 1202. A second hole 1214 may be aligned with hole 1208 in the conductive band 1202. A third hole 1216 may be aligned with hole 1210 in the conductive band 1202. A fourth hole 1228 may be aligned with hole 1224 in the conductive band 1202. A fifth hole 1230 may be aligned with hole 1226 in the conductive band 1202. Another hole (not shown in FIGS. 12A-12B) may be aligned with a hole in the conductive band positioned 180 degrees around the circumference of conductive band 1202 from hole 1206. The conductive band 1102 may include or be formed from stainless steel and the insulating layer 1218 may include or be formed from polyimide. The conductive band 1102 may be attached to the insulating layer 1218 using an adhesive. The insulating layer may prevent contact between the one or more inner electrodes (e.g., 1204a and 1204b) and the conductive band 1202.

The inner electrodes formed by conductive member 1204 and conductive member 1206 are thus spaced apart from the conductive band 1202 by respective gaps formed by a respective one of each of the holes (e.g., holes 1206, 1208, 1210, 1224, 1226, etc.) in the conductive band 1202. Wires 1220 and 1222 may be respectively connected to a supply and return (e.g., positive and negative) terminal of a voltage source. When a voltage is applied across the wire 1220 and wire 1222, an electrical current jumps across a gap formed by each of the respective holes in conductive band 1202 between each of the inner electrodes 1204a-1204c and the conductive band 1202 and between inner electrodes 1206a-1206b and the conductive band 1202, generating a plurality of shock waves. Accordingly, one or more respective shock waves may be generated at shock wave emitters 1213, 1215, 1217, 1219, and 1221 when a voltage is applied across wires 1220 and 1222. One or more shock waves may also be generated at a shock wave emitter positioned circumferentially between emitters 1217 and 1221 (e.g., 180 degrees around the circumference of conductive band 1202 from shock wave emitter 1213) when a voltage is applied across wires 1220 and 1222.

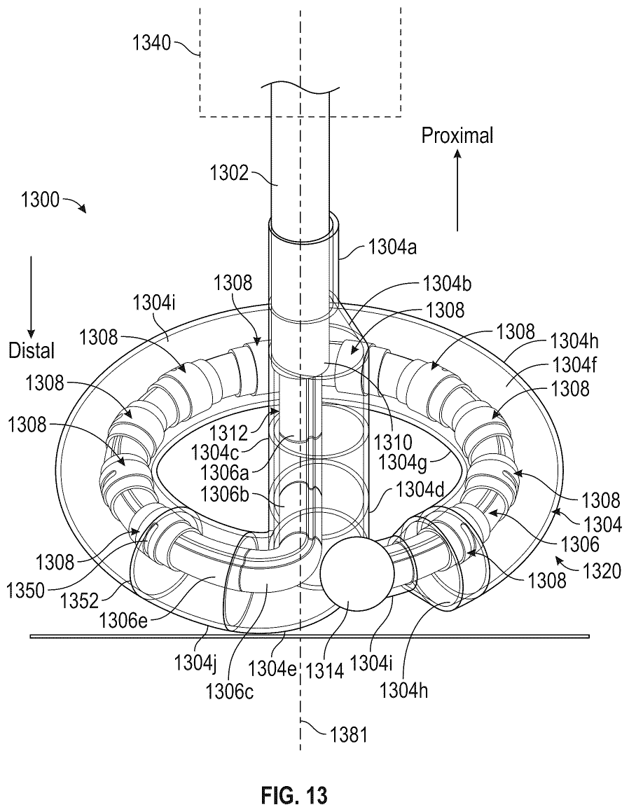

As discussed with reference to FIG. 2, it may be desirable to position shock wave emitters relatively closer to lesions while continuing to allow blood to flow through a body lumen. FIG. 13 illustrates aspects of a catheter 1300 that may share one or more features in common with catheter 100 and/or catheter 200 shown in FIGS. 1 and 2. Aspects of catheter 1300 may include or be used for any of the aspects of system 900 shown in FIG. 9. Catheter 1300 may include a plurality of shock wave emitters 1308, which may include any of the emitter bands and/or wiring configurations described herein with reference to any of FIGS. 1-3D, 4-7, and 10A-12C.

Catheter 1300 may include a catheter body 1302 and an emitter support member 1306. The emitter support member 1306 may extend at least partially within the catheter body 1302 and may extend at least partially distally of a distal end 1310 of the catheter body 1302. The emitter support member may include a pre-formed distal portion 1320 configured coil into an annular loop or “halo” shape around a longitudinal axis 1381 of the catheter body 1302. A plurality of shock wave emitters 1308 may be mounted to the pre-formed distal portion 1320 and the pre-formed distal portion 1320 may enable a user to position the plurality of shock wave emitters 1308 closer to a lesion within a body lumen. For instance, the plurality of shock wave emitters 1308 may be spaced apart from one another along the pre-formed distal portion 1320 such that the plurality of shock wave emitters 1308 can be positioned at a plurality of locations around the inner circumference of a body lumen (e.g., the inner wall of an artery or vein, the annulus of a valve, etc.). The plurality of shock wave emitters 1308 can thus be positioned in close proximity to a lesion to enable a relatively higher amount of force delivered to the lesion.

An enclosure 1304 may be mounted to the catheter body 1302 and/or the emitter support member 1306 and may enclose the plurality of shock wave emitters 1308. Catheter 1300 may be configured such that a conductive fluid can be circulated within the enclosure 1304 to enable shock wave generation. The enclosure may be sealed on one end 1304a to a distal portion of the catheter body 1302 and may be sealed on another end 1304z to a distal portion of the emitter support member 1306. The enclosure 1304 may be configured to enable fluid flow through a body lumen. For instance, the enclosure 1304 may be configured to coil into an annular loop along a path aligned with the emitter support portion. Thus, the enclosure 1304 may define a fluid flow lumen through a center of the annular loop. As illustrated in other examples (e.g., FIG. 1), the enclosure may be an occluding balloon that obstructs fluid flow through the lumen.

The pre-formed distal portion 1320 may be configured to coil such that the plurality of shock wave emitters 1308 positioned at the same longitudinal location relative to a distal end 130 of the catheter body 1302. In some examples, the pre-formed distal portion 1320 may be configured to coil such that the plurality of shock wave emitters 1308 are positioned at different longitudinal locations relative to distal end 1310, such as in a helical pattern (e.g., in embodiments in which the pre-formed distal portion 1320 is formed into a helical structure). Any one or more of the shock wave emitters 1308 may be oriented to direct its shock wave output primarily in a distal direction, primarily in a proximal direction, and/or at an oblique angle therebetween, thereby permitting selective tailoring of the therapeutic shock wave energy to the morphology of the target tissue or lesion. In some examples, one or more of the shock wave emitters 1308 may additionally or alternatively be configured to primarily direct shock wave energy radially outward, (e.g., along vectors extending orthogonally from the longitudinal axis 1381 of the catheter 1300).

The plurality of shock wave emitters 1308 may include slotted emitter bands such as the emitter bands shown in FIGS. 3A-3F. As shown in FIG. 13, a slot 1350 of each emitter band 1352 is oriented such that it faces radially outward away from longitudinal axis 1381. The slot 1350 may form an edge which provides one electrode of an electrode pair (e.g., with a conductive wire positioned radially inward and aligned with the slot 1350). The shock wave energy produced by shock wave emitters 1308 including slotted emitter bands oriented such as the ones depicted in FIG. 13 may thus be directed primarily radially outward. If the slots 1350 were instead oriented to face distally, the shock wave energy may be directed primarily distally from each respective emitter, and so on based on the orientation of the slot. Similarly, for holed emitter bands (i.e., emitter bands having holes instead of elongate slots), the shock wave energy may be directed primarily in the direction that the hole is “facing.”

In some examples, a first subset of shock wave emitters 1308 is configured for primarily distal shock wave emission while a second subset of shock wave emitters 1308 is configured for primarily proximal emission, optionally with intermediate shock wave emitters 1308 oriented radially or at intermediate angular offsets of, for example, 15 degrees-75 degrees relative to the catheter axis, thus enabling a composite 360 degree treatment envelope. At least some of the shock wave emitters 1308 may be individually addressable and at least some of the shock wave emitters 1308 may be configured for simultaneous activation, enabling sequential or simultaneous activation patterns that may correspond to their respective orientations and positions on the emitter support member 1306.

In some examples, the catheter body 1302 is positioned at least partially within an outer elongate sheath 1340. The catheter body 1302 and emitter support member 1306 may be configured to be retracted into and/or deployed from the outer elongate sheath 1340. The enclosure 1304 may also be at least partially retractable into the outer elongate sheath 1340. The pre-formed distal portion 206a may be or include a pre-formed nitinol wire or other shape memory material. When deployed from the elongate sheath 1340, the pre-formed distal portion 206a may naturally coil into an annular loop. The emitter support member 1306 may be fixedly connected to the distal end of the catheter body 1302 such that sliding the catheter body 1302 relative to the elongate sheath 1340 also slides the emitter support member 1306 relative to the elongate sheath 1340. However, in some examples, the emitter support member 1306 may be moveable (e.g., translatable) relative the catheter body 1302 and/or enclosure 1304 such that the emitter support member 1306 can be retracted into and/or deployed via an opening 1312 at the distal end 1310 of the catheter body 1302 and/or may be translated along loop of enclosure 1304. In some examples, the emitter support member 1306 may be rotatable relative to the catheter body 1302, for instance, such that the orientation of the shock wave emitters 1308 can be adjusted (e.g., such that the emitters 1308 can be rotated to position the slots 1350 in a different direction to focus shock wave energy at a desired location).

In some examples, the emitter support member 1306 may include or be connected to an enlarged tip 1314 at an end of the emitter support member 1306. When the emitter support member 1306 is retracted into the catheter body 1302 and/or the outer sheath 1340, the enlarged tip 1314 may act as a stopper abutting the distal end of the catheter body 1302 or the distal end of the outer sheath 1340.

In some examples, the plurality of shock wave emitters are configured to generate shock waves biased in a particular direction (e.g., radially outward, radially inward, distal, and/or proximal) and the plurality of shock wave emitters are positioned relatively closer to a portion of the enclosure that is opposite of the particular direction to prevent damage to the enclosure. In some examples, the plurality of shock wave emitters 1308 may be positioned relatively closer to a portion of the radially inward inner surface 1304g (e.g., closer to the longitudinal axis) of the enclosure 1304 and relatively further from a radially outward inner surface 1304f (e.g., further from the longitudinal axis) of the enclosure 1304. It may be advantageous to position the plurality of shock wave emitters 1308 relatively closer to surface 1304g and relatively further from surface 1304h when the plurality of shock wave emitters 1308 are oriented to direct their shock wave energy primarily radially outward away from the longitudinal axis 1381. Positioning the plurality of shock wave emitters 1308 relatively further from the portion of the inner surface 1304h in such configurations may help to prevent damage to the enclosure that can result from shock wave generation.

In some examples, for instance, when the shock wave emitters 1308 are oriented to direct energy primarily distally, the shock wave emitters 1308 may be positioned relatively more closely to a proximal portion of the inner surface 1304i (a surface closer to the distal end of the catheter body) of the enclosure 1304. Conversely, when the shock wave emitters 1308 are configured for proximally directed emission, they may be relatively closer to a distal portion of the inner surface 1304j (a surface further from the distal end of the catheter body) of the enclosure 1304. Obliquely or radially oriented emitters may be disposed at the corresponding circumferential positions best suited for their respective angular output profiles. Mechanical retention of the emitters on the selected inner surfaces may be achieved by bonding with an adhesive layer applied between the emitter bands 1352 or the emitter support member 1306 and the respective inner surface of enclosure 1304, an over-molding process in which the enclosure 1304 material is cast or thermo-formed around the emitter support member 1306 to capture the emitters in situ, and/or any other retention method. Securing techniques can be employed alone or in combination to maintain precise emitter orientation while preserving enclosure compliance and overall catheter profile.

The pre-formed distal portion may include a plurality of segment that together form the annular loop or “halo” shape. Segment 1304a may extend from the distal end 1310 of the catheter body 1302 in a distal direction along the longitudinal axis 1381 of catheter body 1302. Segment 1306b may extend from segment 1306a and may turn outward away from longitudinal axis 1381 and/or segment 1306a. In some examples, segment 1306b is oriented orthogonally to the longitudinal axis 1381 and/or segment 1306a. In some examples, segment 1306b is oriented at an oblique angle relative to the longitudinal axis 1381 and/or segment 1306a. In some examples, segment 1306b is oriented such that it extends at least partially in a distal direction or a proximal direction. Segment 1306c may extend at an oblique angle from segment 1306b. Segment 1306c may turn clockwise or counterclockwise relative to the longitudinal axis 1381 such that segment 1306c begins to curl around the longitudinal axis 1381 in a circular shape. Segment 1306d may extend from segment 1306c and may form a circle or halo shape extending approximately 360 degrees around the longitudinal axis 1381. In some examples, segment 1306d extends less than 360 degrees around the longitudinal axis 1381. For instance, segment 1306d may extend 60 degrees, 90 degrees, 120 degrees, 180 degrees, 240 degrees, 300 degrees, etc., around the longitudinal axis 1381. In some examples, segment 1306d may extend in a helical pattern in either a proximal or distal direction and may extend for more or less than 360 degrees (e.g., more or less than one full revolution) around the longitudinal axis 1381.

The enclosure 1304 may also include a plurality of pre-formed segments that together form the annular loop or “halo” shape that encloses the halo-shaped emitter support member 1306. Segment 1304a may be a cylindrical segment configured to seal/attach to a distal portion of the catheter body 1302. Segment 1304b may extend distally from segment 1304a and may be tapered relative to segment 1304a such that an average diameter of segment 1304b is larger than an average diameter of segment 1304a. Segment 1304c may extend distally from segment 1304b. Segment 1304c may be of cylindrical shape having an outer diameter equal to a maximum outer diameter of segment 1304b. Segment 1304d may extend from segment 1304c and may turn radially away from longitudinal axis 1381. and/or segment 1304c. In some examples, segment 1304d is oriented orthogonally to the longitudinal axis 1381 and/or segment 1304c. In some examples, segment 1304d is oriented at an oblique angle relative to the longitudinal axis 1381 and/or segment 1304c. In some examples, segment 1304d is oriented such that it extends at least partially in a distal direction or a proximal direction.

Segment 1304e may extend at an oblique angle from segment 1304d. Segment 1304e may turn clockwise or counterclockwise relative to the longitudinal axis 1381 such that segment 1304e begins to curl around the longitudinal axis 1381 in a circular shape. Segment 1304f may extend from segment 1304e and may form a circle or halo shape extending approximately 360 degrees around the longitudinal axis 1381. In some examples, segment 1304f extends less than 360 degrees around the longitudinal axis 1381. For instance, segment 1304 f may extend 60 degrees, 90 degrees, 120 degrees, 180 degrees, 240 degrees, 300 degrees, etc., around the longitudinal axis 1381. In some examples, segment 1304f may extend in a helical pattern in either a proximal or distal direction and may extend for more or less than 360 degrees (e.g., more or less than one full revolution) around the longitudinal axis 1381.

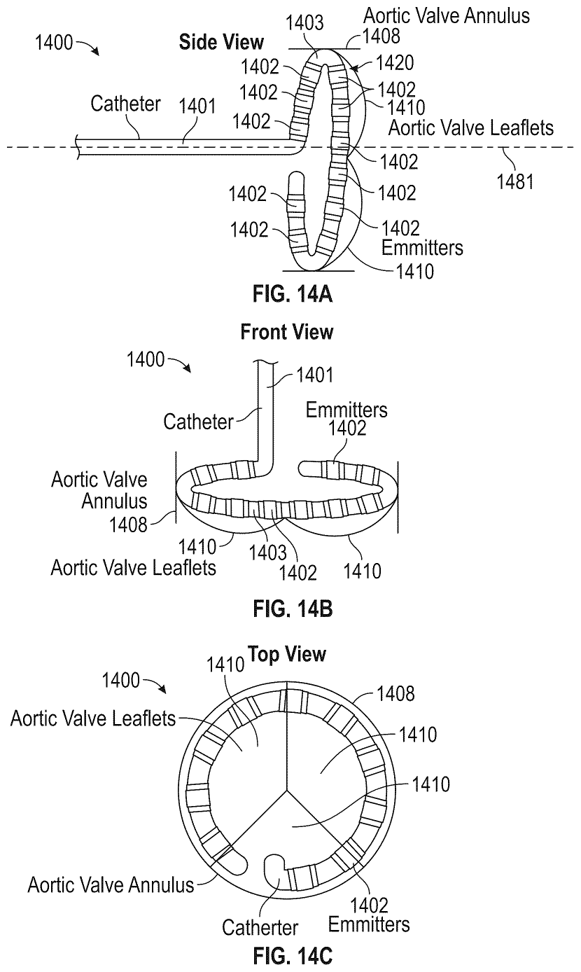

As discussed throughout, the shock wave catheters disclosed herein may be effective for treating lesions (e.g., calcifications) in and around cardiac valves (e.g., aortic valves, mitral valves, etc.). FIGS. 14A-14C illustrate examples of a shock wave catheter 1400 positioned adjacent to a diagrammatic representation of an aortic valve. It should be understood that FIGS. 14A-14C may be representative of other cardiac valves, such as a mitral valve. Catheter 1400 may include any of the aspects of the examples disclosed herein (e.g., catheter 100, 200, 1300, etc.). FIG. 14A illustrates a side view of a catheter 1400 including a catheter body 1401 and an emitter support member 1403. The emitter support member 1403 may be connected to, integral to, positioned within, or otherwise extend from a distal portion of the catheter body 1401. The emitter support member 1403 may include a pre-formed distal portion 1420 configured coil into an annular loop or “halo” shape at least partially around a longitudinal axis 1481 of the catheter body 1401 (in any of the examples disclosed herein, the center of the annular loop may be concentric with or radially offset from the longitudinal axis).

Catheter 1400 may include a plurality of shock wave emitters 1402 positioned one the pre-formed distal portion 1420 of the emitter support member 1403. Catheter 1400, as illustrated in FIGS. 14A-14C is positioned adjacent to an aortic valve annulus 1408 and aortic valve leaflets 1410. The annular loop or “halo” shape of the pre-formed distal portion 1420 is configured to align with and/or conform to the annular shape of the aortic valve annulus 1408 such that the pre-formed distal portion 1420 can be positioned in contact with the aortic valve annulus 1408 and valve leaflets 1410, enabling a user to position the shock wave emitters on the pre-formed distal portion 1420 in close proximity to a lesion on the annulus 1408 and/or leaflets 1410. FIG. 14B illustrates a front view and FIG. 14C illustrates a top view of the catheter 1400 in the same position adjacent to an aortic valve annulus and aortic valve leaflets.

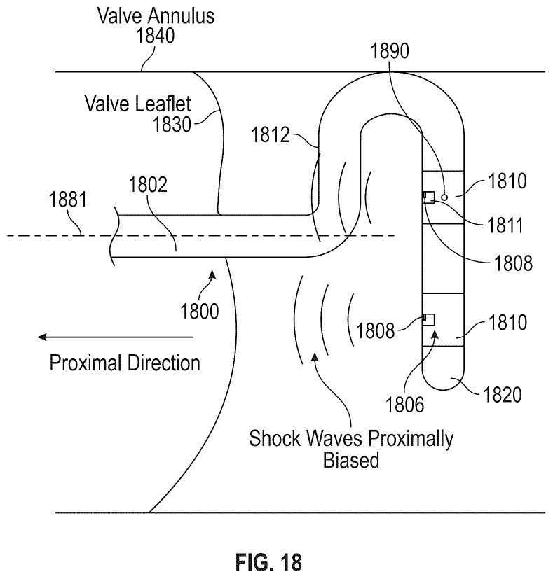

The shock wave emitters included on emitter support member 1403 may be directionally biased to optimize treatment for lesions on different portions of the valve. For instance, the shock wave emitters 1402 may be distally biased to optimize treatment efficacy with respect to lesions on the leaflets 1410 illustrated in FIGS. 14A-14C. The shock wave emitters 1402 may be radially biased to optimize treatment efficacy with respect to lesions on the annulus 1408 illustrated in FIGS. 14A-14C. The shock wave emitters 1402 may be proximally biased to optimize treatment efficacy with respect to lesions on the leaflets 1410 when the catheter 1400 is positioned such that the pre-formed distal portion 1420 is extended through the valve leaflets (e.g., as illustrated in FIG. 18).

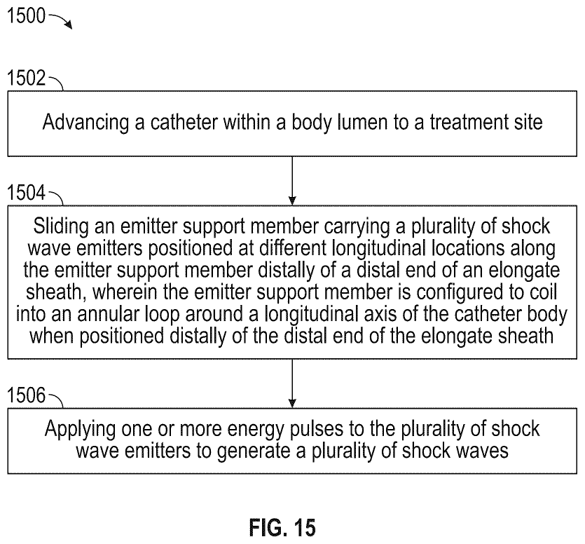

FIG. 15 illustrates a method 1500 of generating shock waves within a body lumen. Method 1400 may be performed, for instance, using any one or more of the aspects of any of the examples disclosed herein (e.g., including any of the aspects described or depicted with reference to FIGS. 1-13). At block 1502, method 1500 may include advancing a catheter within a body lumen to a treatment site. The target treatment site may be a cardiac valve (e.g., the aortic valve depicted in FIGS. 14A-14C). The cardiac valve may be a tricuspid valve, mitral valve, pulmonary valve, or aortic valve.

At block 1504, method 1500 may include sliding an emitter support member carrying a plurality of shock wave emitters positioned at different longitudinal locations along the emitter support member distally of a distal end of an elongate sheath. The emitter support member may be configured to coil into an annular loop around a longitudinal axis of the catheter body when positioned distally of the distal end of the elongate sheath (e.g., as described with reference to FIGS. 1, 2, and 13). Thus, sliding the emitter support member distally of a distal end of the elongate sheath may cause the plurality of shockwave emitters to move radially away from a longitudinal axis of the catheter body. The plurality of shock wave emitters may be spaced apart from one another along a length of the emitter support member and/or may be oriented such that an electrode pair of at least a subset of the plurality of shock wave emitters is positioned adjacent to and facing a base of a valve leaflet or a valve annulus. After sliding the emitter support member distally, a user may manipulate the catheter to position the plurality of shockwave emitters such that a plurality of emitters are adjacent to and facing an annulus of the valve and/or one or more leaflets of the valve.

In some examples, the annular loop of the emitter support member is a closed loop. In some examples, an enclosure enclosing the annular loop of the emitter support member may form an enclosed loop (e.g., a closed ring/halo) and/or may be an occluding balloon. Such a closed loop emitter support member and/or enclosure may be beneficial, for instance, when the target treatment site is a sub-annular valve. Sub-annular valves may have chordae (e.g., chordae tendineae) that may get caught on an open loop (e.g., as illustrated in FIGS. 2 and 13). A closed loop and/or occluding balloon configuration may be less prone to getting caught on such chordae.

At block 1506, method 1500 may include applying one or more energy pulses to the plurality of shock wave emitters to generate a plurality of shock waves. After shock wave treatment, the plurality of shock wave emitters may be retracted into the catheter body and the catheter may be removed from the body lumen. In some examples, the plurality of shock wave emitters may be oriented such that shock wave energy is directed primarily distally, primarily proximally, and/or primarily radially. In some examples, one or more shock wave emitters may be oriented such that shock wave energy is directed primarily distally, one or more other shock wave emitters may be oriented to direct shock wave energy primarily proximally, and one or more other shock wave emitters may be oriented to direct shock wave energy primarily radially. In some examples, the plurality of shock wave emitters are oriented such that sonic output produced during shock wave generation is directed in a primarily proximal direction, and the the catheter may be advanced across a valve annulus such that the treatment site is positioned proximally of the plurality of shock wave emitters, thus enabling a user to easily position the emitters in close proximity to a difficult to access treatment site.

After generating the plurality of shock waves, a user may retract the plurality of shock wave emitters into the elongate sheath. In some examples, after treating the target treatment site (e.g., a cardiac valve) with shock wave energy, for instance, to break up calcifications or otherwise modify a lesion, additional or different treatment steps may be taken according to method 1500. For instance, the method may include implanting an aortic valve implant after generating the plurality of shock waves. Additionally, or alternatively, the method may include positioning a valvuloplasty balloon at across a valve annulus of the treatment site after generating the plurality of shock waves and inflating the balloon to widen the valve annulus. In some examples, the plurality of shock wave emitters are simply repositioned to target a different portion of the treatment site, and an additional plurality of shock waves are generated.

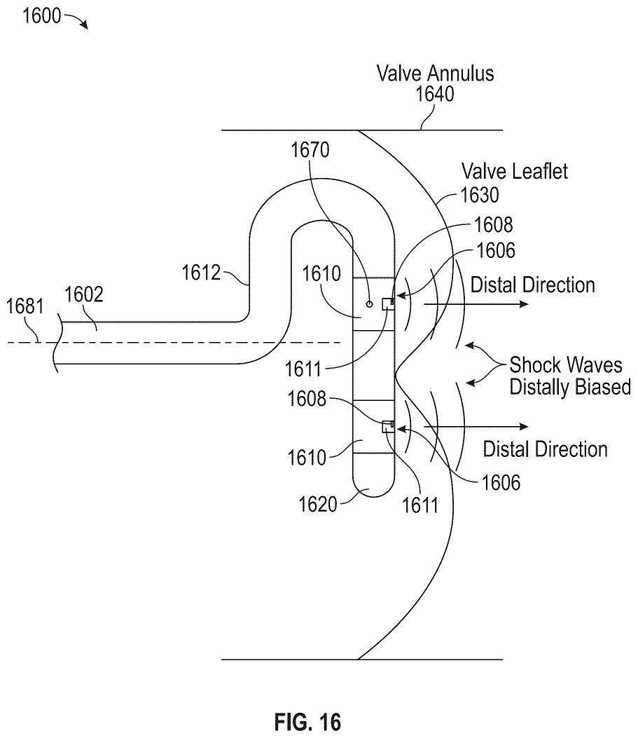

FIG. 16 shows a schematic diagram illustrating aspects of a shock wave catheter 1600 having distally biased shock wave emitters 1606 positioned adjacent to a cardiac valve (e.g., an aortic valve). FIG. 16 shows a side view of catheter 1600. The catheter 1600 shown in FIG. 16 may include one or more features in common with any of the catheters described herein (e.g., catheter 100, 200, 1300, etc.). Catheter 1600 may include a catheter body 1602 and an emitter support member 1612. The emitter support member 1612 may be connected to, integral to, positioned within, or otherwise extend from a distal portion of the catheter body 1602. The emitter support member 1612 may include a pre-formed distal portion 1620 configured coil into an annular loop or “halo” shape around a longitudinal axis 1681 of the catheter body 1602 (in any of the examples disclosed herein, the center of the annular loop may be concentric with or radially offset from the longitudinal axis).

A plurality of shock wave emitters 1606 may be mounted to the pre-formed distal portion 1620 and the pre-formed distal portion 1620 may enable a user to position the plurality of shock wave emitters 1606 closer to a lesion within a body lumen. For instance, as illustrated in FIG. 16, the plurality of shock wave emitters 1606 may be spaced apart from one another along the pre-formed distal portion 1620 such that the plurality of shock wave emitters 1606 can be positioned at a plurality of locations circumferentially spaced around leaflets of a cardiac valve. The plurality of shock wave emitters 1606 can thus be positioned in close proximity to a lesion on one or more valve leaflets at a plurality of circumferential locations to enable a relatively higher amount of force delivered to the lesion.

As noted above, the shock wave emitters 1606 may be distally biased such that shock waves generated using emitter 1606 are directed primarily in the distal direction. The biasing may be accomplished based at least in part on the positioning of the electrode pairs forming each shock wave emitter on the catheter 1600. Each shock wave emitter 1606 may include an electrode pair formed by an inner conductor 1608 and an emitter band 1610 spaced apart by a spark gap 1611. The electrode pair of each shock wave emitter 1606 may be positioned at a circumferential location of the emitter band circumferentially offset by between 0 and 180 degrees (e.g., 90 degrees) from a circumferential location 1670 of the emitter band. Circumferential location 1670 of the emitter band may be positioned 180 degrees offset from a corresponding circumferential location of the emitter band oriented radially inwardly toward a center of the annular loop formed by the pre-formed distal portion 1620. Thus, the electrode pairs of the respective shock wave emitters 1606 may be positioned at a circumferential location of the emitter band 1610 that faces distally and shock waves generated by the respective emitters 1606 may propagate primarily distally toward the valve leaflets.

During use, catheter 1600 may be positioned adjacent to a cardiac valve (e.g., an aortic valve, mitral valve, tricuspid valve). The pre-formed distal portion 1612 (or an enclosure surrounding the pre-formed distal portion 1612, such as enclosure 1304) may be positioned in contact with the valve leaflets 1630 and/or valve annulus 1640. The pre-formed distal portion may be configured such that it conforms to the annular shape of the valve annulus and is positioned in contact with the valve annulus along its inner circumference. A user may activate shock wave emitters (e.g., by supplying an energy pulse) to generate a plurality of shock waves to modify, break up, or otherwise disrupt/soften a lesion on the valve leaflets 1630 and/or annulus 1640. The distally biased emitters illustrated in FIG. 16 may enable optimal treatment for lesions on the valve leaflets 1630, which are positioned adjacent the distal facing surface of the emitter bands when the catheter 1600 is positioned adjacent to the valve.

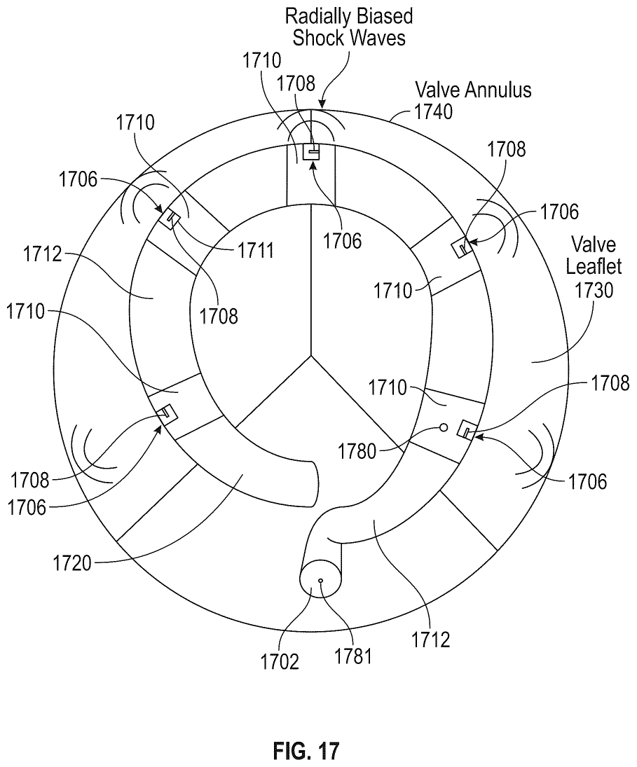

FIG. 17 shows a schematic diagram illustrating aspects of a shock wave catheter 1700 having radially biased shock wave emitters 1706 positioned adjacent to a cardiac valve (e.g., an aortic valve). FIG. 17 depicts a view of catheter 1700 from a proximal end of the catheter. The catheter 1700 shown in FIG. 17 may include one or more features in common with any of the catheters described herein (e.g., catheter 100, 200, 1300, etc.). Catheter 1700 may include a catheter body 1702 and an emitter support member 1712. The emitter support member 1712 may be connected to, integral to, positioned within, or otherwise extend from a distal portion of the catheter body 1702. The emitter support member 1712 may include a pre-formed distal portion 1720 configured coil into an annular loop or “halo” shape around a longitudinal axis 1781 of the catheter body 1702 (in any of the examples disclosed herein, the center of the annular loop may be concentric with or radially offset from the longitudinal axis).

A plurality of shock wave emitters 1706 may be mounted to the pre-formed distal portion 1720 and the pre-formed distal portion 1720 may enable a user to position the plurality of shock wave emitters 1706 closer to a lesion within a body lumen. For instance, as illustrated in FIG. 17, the plurality of shock wave emitters 1706 may be spaced apart from one another along the pre-formed distal portion 1720 such that the plurality of shock wave emitters 1706 can be positioned at a plurality of locations circumferentially spaced around an annulus of a cardiac valve. The plurality of shock wave emitters 1706 can thus be positioned in close proximity to a lesion on the annulus at a plurality of circumferential locations to enable a relatively higher amount of force delivered to the lesion.

As noted above, the shock wave emitters 1706 may be radially biased such that shock waves generated using emitter 1706 are directed primarily in the radial direction (outward toward the annulus 1740). The biasing may be accomplished based at least in part on the positioning of the electrode pairs forming each shock wave emitter on the catheter 1700. Each shock wave emitter 1706 may include an electrode pair formed by an inner conductor 1708 and an emitter band 1710 spaced apart by a spark gap 1711. The electrode pair of each shock wave emitter 1706 may be positioned at a circumferential location of the emitter band circumferentially offset by between 0 and 180 degrees (e.g., 90 degrees) from a circumferential location 1780 of the emitter band. Circumferential location 1780 of the emitter band may be positioned 180 degrees offset from a corresponding circumferential location of the emitter band oriented distally—thus, circumferential location 1780 may be oriented toward a proximal end of the catheter body 1702. Thus, the electrode pairs of the respective shock wave emitters 1706 may be positioned at a circumferential location of the emitter band 1710 that faces radially outward (e.g., configured to face toward a valve annulus 1740) and shock waves generated by the respective emitters 1706 may propagate primarily radially toward the valve annulus 1740.

During use, catheter 1700 may be positioned adjacent to a cardiac valve (e.g., an aortic valve, mitral valve, tricuspid valve). The pre-formed distal portion 1712 (or an enclosure surrounding the pre-formed distal portion 1712, such as enclosure 1304) may be positioned in contact with the valve leaflets 1730 and/or valve annulus 1740. The pre-formed distal portion may be configured such that it conforms to the annular shape of the valve annulus 1740 and is positioned in contact with the valve annulus along its inner circumference. A user may activate shock wave emitters (e.g., by supplying an energy pulse) to generate a plurality of shock waves to modify, break up, or otherwise disrupt/soften a lesion on the valve leaflets 1730 and/or annulus 1740. The radially biased emitters illustrated in FIG. 17 may enable optimal treatment for lesions on the valve annulus 1740, which are positioned adjacent the radially facing surface of the emitter bands when the catheter 1700 is positioned adjacent to the valve.

FIG. 18 shows a schematic diagram illustrating aspects of a shock wave catheter 1800 having proximally biased shock wave emitters 1806 positioned adjacent to a cardiac valve (e.g., an aortic valve). The catheter 1800 shown in FIG. 18 may include one or more features in common with any of the catheters described herein (e.g., catheter 100, 200, 1300, etc.). Catheter 1800 may include a catheter body 1802 and an emitter support member 1812. The emitter support member 1812 may be connected to, integral to, positioned within, or otherwise extend from a distal portion of the catheter body 1802. The emitter support member 1812 may include a pre-formed distal portion 1820 configured coil into an annular loop or “halo” shape around a longitudinal axis 1881 of the catheter body 1802 (in any of the examples disclosed herein, the center of the annular loop may be concentric with or radially offset from the longitudinal axis).

A plurality of shock wave emitters 1806 may be mounted to the pre-formed distal portion 1820 and the pre-formed distal portion 1820 may enable a user to position the plurality of shock wave emitters 1806 closer to a lesion within a body lumen. For instance, as illustrated in FIG. 18, the plurality of shock wave emitters 1806 may be spaced apart from one another along the pre-formed distal portion 1820 such that the plurality of shock wave emitters 1808 can be positioned at a plurality of locations circumferentially spaced around leaflets 1830 of a cardiac valve. The plurality of shock wave emitters 1806 can thus be positioned in close proximity to a lesion on one or more valve leaflets 1830 at a plurality of circumferential locations to enable a relatively higher amount of force delivered to the lesion.

As noted above, the shock wave emitters 1806 may be proximally biased such that shock waves generated using emitter 1806 are directed primarily in the proximal direction (e.g., toward a proximal end of catheter 1800). The biasing may be accomplished based at least in part on the positioning of the electrode pairs forming each shock wave emitter on the catheter 1800. Each shock wave emitter 1806 may include an electrode pair formed by an inner conductor 1808 and an emitter band 1810 spaced apart by a spark gap 1811. The electrode pair of each shock wave emitter 1806 may be positioned at a circumferential location of the emitter band circumferentially offset by between 0 and 180 degrees (e.g., 90 degrees) from a circumferential location 1890 of the emitter band. Circumferential location 1890 of the emitter band may be positioned 180 degrees offset (proximally) from a corresponding circumferential location of the emitter band oriented radially inwardly toward a center of the annular loop formed by the pre-formed distal portion 1820. Circumferential location 1890 may be positioned at a circumferential location oriented radially outward (e.g., toward valve annulus 1840). Thus, the electrode pair of the respective shock wave emitters 1806 may be positioned at a circumferential location of the emitter band 1810 that faces proximally and shock waves generated by the respective emitters 1806 may propagate primarily proximally toward a proximal end of the catheter body 1802.

During use, catheter 1800 may be advanced across a valve annulus 1840 such that the pre-formed distal portion 1820 is advanced through the valve. The pre-formed distal portion 1820 can then be positioned adjacent to a cardiac valve (e.g., an aortic valve, mitral valve, tricuspid valve) with its proximal facing side adjacent to the valve leaflets 1830. The pre-formed distal portion 1812 (or an enclosure surrounding the pre-formed distal portion 1812, such as enclosure 1304) may be positioned in contact with the valve leaflets 1830 and/or valve annulus 1840. The pre-formed distal portion 1820 may be configured such that it conforms to the annular shape of the valve annulus and is positioned in contact with the valve annulus 1840 and/or leaflets 1830. A user may activate shock wave emitters (e.g., by supplying an energy pulse) to generate a plurality of shock waves to modify, break up, or otherwise disrupt/soften a lesion on the valve leaflets 1830 and/or annulus 1840. The proximally biased emitters illustrated in FIG. 18 may enable optimal treatment for lesions on the valve leaflets 1830 on the distal surface of the leaflets, which can be positioned adjacent the proximal facing surface of the emitter bands when the catheter 1800 is advanced through the valve and positioned such that the proximal-facing surface of the pre-formed distal portion 1820 is positioned adjacent to the valve leaflets 1830.

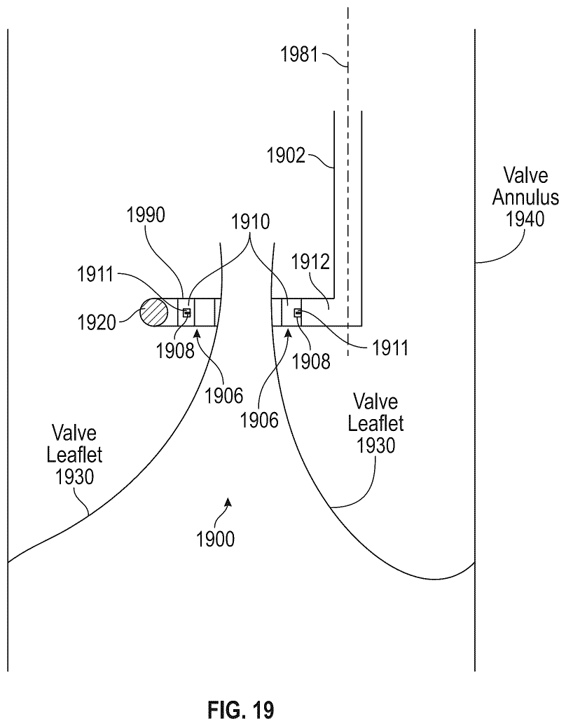

FIG. 19 shows a schematic diagram illustrating aspects of a shock wave catheter 1800 having radially inwardly biased shock wave emitters 1906 positioned adjacent to a cardiac valve (e.g., an aortic valve). The catheter 1900 shown in FIG. 19 may include one or more features in common with any of the catheters described herein (e.g., catheter 100, 200, 1300, etc.). Catheter 1900 may include a catheter body 1902 and an emitter support member 1912. The emitter support member 1912 may be connected to, integral to, positioned within, or otherwise extend from a distal portion of the catheter body 1902. The emitter support member 1912 may include a pre-formed distal portion 1920 configured coil into an annular loop or “halo” shape around a longitudinal axis 1981 of the catheter body 1902 (in any of the examples disclosed herein, the center of the annular loop may be concentric with or radially offset from the longitudinal axis).

A plurality of shock wave emitters 1906 may be mounted to the pre-formed distal portion 1920 and the pre-formed distal portion 1920 may enable a user to position the plurality of shock wave emitters 1906 closer to a lesion within a body lumen. For instance, as illustrated in FIG. 19, the plurality of shock wave emitters 1906 may be spaced apart from one another along the pre-formed distal portion 1920 such that the plurality of shock wave emitters 1908 can be positioned at a plurality of locations circumferentially spaced around leaflets 1930 of a cardiac valve and at least partially circumscribing the leaflets 1930. The plurality of shock wave emitters 1906 can thus be positioned in close proximity to a lesion on one or more valve leaflets 1930 at a plurality of circumferential locations to enable a relatively higher amount of force delivered to the lesion.

As noted above, the shock wave emitters 1906 may be inwardly biased such that shock waves generated using emitter 1906 are directed primarily in the radially inward direction (e.g., the leaflets 1930 shown in FIG. 19). The biasing may be accomplished based at least in part on the positioning of the electrode pairs forming each shock wave emitter 1906 on the catheter 1900. Each shock wave emitter 1906 may include an electrode pair formed by an inner conductor 1908 and an emitter band 1910 spaced apart by a spark gap 1911. The electrode pair of each shock wave emitter 1906 may be positioned at a circumferential location of the emitter band circumferentially offset by between 0 and 180 degrees (e.g., 90 degrees) from a circumferential location 1990 of the emitter band. Circumferential location 1990 may be positioned at a circumferential location oriented proximally (e.g., toward a proximal end of catheter 1900). Thus, the electrode pairs of the shock wave emitters 1906 may be positioned at a circumferential location of the emitter band 1910 that faces radially inward and shock waves generated by the respective emitters 1906 may propagate primarily inward toward a center of the loop formed by pre-formed distal portion 1920.

During use, catheter 1900 may be positioned such that the pre-formed distal portion 1920 circumscribes at least a portion of the valve leaflets 1930 of a cardiac valve. The pre-formed distal portion 1920 (or an enclosure surrounding the pre-formed distal portion 1920, such as enclosure 1304) may be positioned in contact with the valve leaflets 1930 and/or valve annulus 1940. The pre-formed distal portion 1920 may be configured such that it conforms to the annular shape of the valve annulus and is positioned in contact with the valve annulus 1940 and/or leaflets 1930. A user may activate shock wave emitters (e.g., by supplying an energy pulse) to generate a plurality of shock waves to modify, break up, or otherwise disrupt/soften a lesion on the valve leaflets 1930. The radially inwardly biased emitters illustrated in FIG. 19 may enable optimal treatment for lesions on the valve leaflets 1930, which can be positioned adjacent the inward facing portion of the emitter bands 1910 when the catheter 1900 is positioned such that the pre-formed distal portion 1920 at least partially circumscribes the leaflets 1930.

As described throughout, exemplary catheters may include pre-formed distal portions carrying a plurality of shock wave emitters. In some examples, it may be desirable to configure a catheter such that a pre-formed member is separate from a member carrying the shock wave emitters. For instance, it may be desirable to enable a user to slide the member carrying the shock wave emitters relative to a pre-formed member to enable a user to target different areas of a treatment site. It may additionally, or alternatively, be desirable to configure the pre-formed member to fold into an outer sheath and unfold from the outer sheath as opposed to straightening when pulled into an outer sheath and spiraling into a loop when deployed from the sheath. A pre-formed distal member that folds when retracted into an outer sheath and unfolds when deployed from the outer sheath may be less prone to becoming entangled with chordae near a valve. The pre-formed loop may additionally, or alternatively, be a closed loop to further mitigate risk of catching on the chordae.

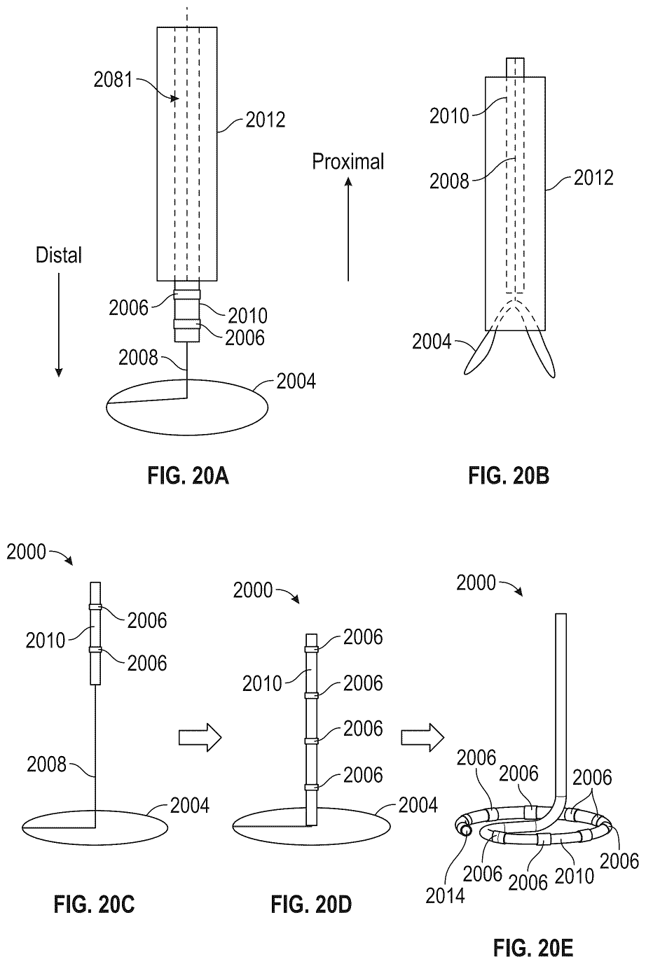

FIGS. 20A-20B illustrate aspects of a catheter 2000 that includes an outer sheath 2012, an elongate member 2010 positioned at least partially within the outer sheath 2012 and an inner member 2008 that includes a pre-formed distal portion 2004 deployable from the outer sheath 2012. Catheter 2000 may include or be used for aspects of any of the catheters disclosed herein. The inner member 2008 may be positioned at least partially within the elongate member 2010 and the inner member 2004 may be configured such that when it is deployed from the outer sheath 2012, the pre-formed distal portion 2004 unfolds into an annular loop around a longitudinal axis 2081 of the outer sheath 2012. A plurality of shock wave emitters 2006 may be positioned on a distal portion the elongate member.

The elongate member 2010 may be translatable relative to the inner member 2008 and pre-formed distal portion 2004 between an extended position and a retracted position. The distal portion of elongate member 2010 may conform to the annular loop of the pre-formed distal portion in the extended position. For instance, the elongate member 2010 may include a central lumen 2014 and the inner member 2008 may extend within the central lumen 2014 such that the elongate member 2010 can be translated along the inner member 2008. When the pre-formed distal portion 2004 is deployed from the outer sheath 2012 (e.g., as shown in FIG. 20A), the elongate member 2010 may be translated distally relative to outer sheath 2012 along the inner member 2008 to position the shock wave emitters 2006 adjacent to a lesion. The elongate member may be translated distally along a portion of the inner member 2008, as shown between FIGS. 20C and 20D, and then may turn and follow the annular loop of the pre-formed distal portion 2004 of inner member 2008, as depicted by the change in position between FIGS. 20D and 20E. Thus, the elongate member 2010 may be gradually advanced along inner member 2008 until a distal portion of the elongate member 2010 carrying shock wave emitters 2006 takes on the annular loop shape of the pre-formed distal portion 2004 (e.g., as shown in the transition from FIG. 20C to FIG. 20E. The pre-formed distal portion 2004 may be retractable into the outer sheath 2012 and may be configured such that when the pre-formed distal portion 2004 is retracted into the outer sheath 2012, it folds over on itself (e.g., in half) into a folded configuration, for instance, as shown in FIG. 20B.

In some examples, the elongate member 2010 is bonded (e.g., fixedly attached) to the inner member 2008. A distal portion of the elongate member 2010 may thus be bonded to the pre-formed distal portion 2008 and configured to unfold into the annular loop when the pre-formed distal portion 2004 is deployed from the outer sheath 2012. The elongate member 2010 may be configured to move in tandem with inner member 2008 such that the elongate member 2010 and the pre-formed distal portion 2004 of inner member 2008 fold into the folded configuration when retracted into the outer sheath 2012 and unfold when deployed from outer sheath 2012. In some examples, the pre-formed distal portion 2004 may form a closed annular loop (e.g., a circle, an ellipse, a semicircle, etc.) around the longitudinal axis 2081. In some examples, the pre-formed distal portion 2004 may form an open loop (e.g., a circle, an ellipse, a semicircle, etc., having a free end) around the longitudinal axis 2081. In further examples, the pre-formed distal portion 2004 can form a shape sized and configured to comport to or match with the annular region of a given heart valve. The inner member 2008 (including pre-formed distal portion 2004) may be formed from a shape memory material, such as nitinol or copper-based alloys. In some examples, an enclosure (e.g., such as enclosure 204) may be sealed to a distal end of the elongate member 2010 and/or the inner member 2008. The enclosure (e.g., enclosure 204) may be configured to unfold into a corresponding annular loop in the deployed position when the enclosure is positioned distal of a distal end of the outer sheath 2012.

The elements and features of the example catheters and catheter systems illustrated throughout this specification and drawings may be rearranged, recombined, and modified without departing from the present disclosure. For instance, the number, placement, and spacing of shock wave generating regions or emitters can be modified and the number, placement, and spacing of the enclosures of catheters can be modified without departing from the present disclosure.

Although the catheter devices described herein have been discussed primarily in the context of treating coronary occlusions, such as lesions in vasculature, the catheter devices described herein can be used for a variety of occlusions, such as occlusions in the peripheral vasculature (e.g., above-the-knee, below-the-knee, iliac, carotid, etc.). For further examples, various embodiments may be used for treating soft tissues, such as cancer and tumors (i.e., non-thermal ablation methods), blood clots, fibroids, cysts, organs, scar and fibrotic tissue removal, or other tissue destruction and removal treatments. Electrode assembly and catheter designs could also be used for neurostimulation treatments, targeted drug delivery, treatments of tumors in body lumens (e.g., tumors in blood vessels, the esophagus, intestines, stomach, or vagina), wound treatment, non-surgical removal, and destruction of tissue, or used in place of thermal treatments or cauterization for venous insufficiency and fallopian ligation (i.e., for permanent female contraception).

In one or more examples, the electrode assemblies and, catheters described herein could also be used for tissue engineering methods, for instance, for mechanical tissue decellularization to create a bioactive scaffold in which new cells (e.g., exogenous and endogenous cells) can replace the old cells; introducing porosity to a site to improve cellular retention, cellular infiltration/migration, and diffusion of nutrients and signaling molecules to promote angiogenesis, cellular proliferation, and tissue regeneration similar to cell replacement therapy. Such tissue engineering methods may be useful for treating ischemic heart disease, fibrotic liver, fibrotic bowel, and traumatic spinal cord injury (SCI). For instance, for the treatment of spinal cord injury, the devices and assemblies described herein could facilitate the removal of scarred spinal cord tissue, which acts like a barrier for neuronal reconnection, before the injection of an anti-inflammatory hydrogel loaded with lentivirus to genetically engineer the spinal cord neurons to regenerate.

It will be understood that the foregoing is only illustrative, and that various modifications, alterations and combinations can be made by those skilled in the art without departing from the scope and spirit of the disclosure. Any of the variations of the various catheters disclosed herein can include features described by any other catheters or combination of catheters herein. Furthermore, any of the methods can be used with any of the catheters disclosed. Accordingly, it is not intended that the systems, catheters, and methods described herein be limited, except as by the appended claims.

### Clauses

1. A catheter for treating calcified tissue in a body with shock waves, the catheter comprising:
   an elongate shaft extending from a proximal region to a distal region of the catheter and including a fluid lumen and a central axis;
   an enclosure located at the distal region of the catheter, the enclosure in fluid communication with a fluid source via the fluid lumen;
   a plurality of shock wave emitters enclosed within the enclosure and radially offset from the central axis, each of the shock wave emitters comprising at least one electrode pair configured to emit at least one shock wave radially outward from the central axis when a voltage pulse is applied across the electrode pair; and
   a conductive member electrically connected to the plurality of shock wave emitters from a voltage pulse generator.
2. The catheter of clause 1, wherein the emitter comprises a conductive band and each electrode pair comprises a first electrode and a second electrode, the first electrode formed, at least in part, by a conductive surface of the conductive band, the conductive surface located along an elongate slot extending circumferentially along the conductive band.
3. The catheter of clause 2, wherein the elongate slot extends 30 degrees to 330 degrees around a circumference of the conductive band.
4. The catheter of clause 3, wherein the elongate slot extends 60 degrees to 120 degrees around a circumference of the conductive band.
5. The catheter of any one of clauses 2-4, wherein the second electrode comprises a conductive surface radially inward of the conductive band that is electrically connected to the conductive member.
6. The catheter of clause 5, wherein the conductive surface is located on an inner band in contact with the conductive member.
7. The catheter of any one of clauses 5-6, wherein the conductive surface is located on a distal region of the conductive member.
8. The catheter of any one of clauses 1-7, wherein the plurality of shock wave emitters are connected to each other in series and the catheter comprises a return wire that is electrically connected to the voltage pulse generator.
9. The catheter of any one of clauses 1-8, wherein the at least one electrode pair comprises a first electrode having a first surface area and a second electrode having a second surface area greater than the first surface area and wherein the first electrode is configured to act as a cathode and the second electrode is configured to act as an anode.
10. The catheter of any one of clauses 1-9, wherein the emitter comprises a conductive band having an aperture and wherein the at least one electrode pair comprises a first electrode formed by a distal surface of the conductive member and a second electrode formed by a conductive surface defined by the conductive band.
11. The catheter of any one of clauses 1-10, wherein the enclosure comprises an expanded diameter of 10 mm to 50 mm.
12. The catheter of any one of clauses 1-11, wherein the enclosure, in an expanded state, comprises a diameter greater than an axial length.
13. The catheter of clauses 11 or 12, wherein the enclosure includes a guidewire lumen.
14. The catheter of any one of clauses 1-13, wherein the enclosure comprises a disc-shape and the plurality of shock wave emitters are spaced within the enclosure.
15. The catheter of clause 14, wherein the shock wave emitters are evenly spaced within the enclosure.
16. The catheter of any one of clauses 1-15, wherein the enclosure comprises a cylindrical coil shape defining an opening therethrough for blood to flow.
17. The catheter of any one of clauses 1-16, wherein the shock wave emitters are mounted along a distal region of an elongate member that, in a first configuration, is received within the elongate shaft and, in a second configuration, is positioned within the enclosure.
18. The catheter of clause 17, wherein the elongate member comprises, at its distal region a shape memory material.
19. The catheter of clause 18, wherein the shape memory material comprises nitinol.
20. The catheter of any one of clauses 1-19, wherein the shock wave emitters are mounted along an elongate member that is slidably received in the elongate shaft.
21. A catheter system for treating calcified tissue in a body with shock waves, the system comprising:
   a voltage pulse generator; and
   a catheter comprising:
      a first conductive member;
      a second conductive member;
      a third conductive member;
      a first electrode pair comprising a first cathode and a first anode, where the first anode is formed from a more durable material than the first cathode;
      a second electrode pair including a second cathode and a second anode, where the second anode is formed from a more durable material than the second cathode,
   where current travels via the first conductive member from the voltage pulse generator to the first cathode, arcs from the first cathode to the first anode generating a first shock wave, travels via the second conductive member to the second cathode to the second anode generating a second shock wave, and returns via the third conductive member to the voltage pulse generator.
22. The catheter of clause 21, wherein the first electrode pair and the second electrode pair are circumferentially distributed about a central axis of the catheter and the first electrode pair and the second electrode pair are configured such that the first and second shock waves propagate radially away from the central axis.
23. A catheter for treating calcified tissue in a body with shock waves, the catheter comprising:
   an emitter band having a slot that extends 30 degrees to 330 degrees around a circumference of the emitter band, where a conductive surface of the slot forms at least a part of at least a first electrode of an electrode pair;
   a conductive member having a conductive surface that forms at least a part of a second electrode of the electrode pair, where the catheter is configured to generate a shock wave when a voltage pulse is applied across the electrode pair; and an enclosure that is fillable with fluid.
24. The catheter of clause 23, wherein the slot comprises two slots.
25. The catheter of any one of clauses 23-24, wherein the catheter includes a central axis and the electrode pair is configured such that the shock wave propagates radially away from the central axis.
26. A catheter for treating calcified tissue in a body, the catheter comprising:
   an elongate shaft; and
   at least two shock wave emitters that, in a first configuration are received in the
   elongate shaft and, in a second configuration, are outside of the elongate shaft and
   spaced away from a longitudinal central axis of the elongate shaft.
27. A shock wave emitter band for a shock wave catheter, the emitter band comprising a body and a slot that extends circumferentially around at least part of the body.
28. An electrohydraulic method of generating shock waves, the method comprising:
   delivering a voltage pulse to a catheter,
   where the voltage pulse is delivered via a first conductive member that includes a first conductive surface spaced from a first emitter band by a first gap,
   where when the voltage pulse is applied across the first gap, a first shock wave is generated,
   where after the voltage pulse is applied across the first gap, the voltage pulse is delivered to a second emitter band by a second conductive member, which is in contact with the first emitter band and includes a second conductive surface spaced from the second emitter band by a second gap,
   where when the voltage pulse is applied across the second gap, a second shock wave is generated.
29. The catheter of any of the preceding clauses, wherein the calcified tissue is located in the heart.
30. The catheter of any of the preceding clauses, wherein the calcified tissue is at or proximate an aortic valve.
31. A catheter for generating shock waves, the catheter comprising:
   a catheter body;
   a conductive band mounted to the catheter body, wherein the conductive band comprises at least one elongate slot that extends at least partially around the circumference of the conductive band; and
   at least one electrode positioned radially inward of the conductive band and aligned with the at least one elongate slot, wherein the at least one electrode and the conductive band form an electrode pair of a shock wave emitter.
32. The catheter of clause 31, wherein the elongate slot extends more than 180 degrees around the circumference of the conductive band.
33. The catheter of any one of clauses 31-32, wherein the elongate slot extends helically around the conductive band.
34. The catheter of clause 33, wherein a first electrode of the at least one electrode is positioned proximally of a second electrode of the at least one electrode, and wherein each of the first electrode and the second electrode are aligned with the elongate slot.
35. The catheter of clause 34, wherein the first electrode is spaced apart from the second electrode around the circumference of the conductive band by 180 degrees.
36. The catheter of any one of clauses 31-35, comprising an insulating layer positioned between the conductive band and the at least one electrode, wherein the insulating layer comprises at least one elongate slot aligned with the at least one elongate slot of the conductive band.
37. A catheter for generating shock waves, the catheter comprising:
   a catheter body;
   a conductive band mounted to the catheter body, wherein a plurality of holes are formed into the conductive band at a plurality of circumferential locations of the conductive band;
   a conductive member positioned radially inward of the conductive band, wherein:
   a first portion of the conductive member is aligned with a first hole of the plurality of holes, wherein the first portion of the conductive member and the conductive band form an electrode pair of a first shock wave emitter; and
   a second portion of the conductive member is aligned with a second hole of the plurality of holes, wherein the second portion of the conductive member and the conductive band form an electrode pair of a second shock wave emitter.
38. The catheter of clause 37, wherein the conductive band comprises at least three holes and the conductive member is aligned with the at least three holes, thereby forming at least three shock wave emitters.
39. The catheter of any one of clauses 37-38, comprising a second conductive member positioned radially inward of the conductive band and spaced apart from the conductive member.
40. The catheter of clause 39, wherein a first end of the conductive member is spaced apart from a first end of the second conductive member by between 5 degrees and 45 degrees around the circumference of the conductive band.
41. The catheter of clause 40, wherein a second end of the conductive member is spaced apart from a second end of the second conductive member by between 5 degrees and 45 degrees around the circumference of the conductive band.
42. The catheter of any one of clauses 39-41, wherein the first conductive member and the second conductive member are semi-cylindrical.
43. The catheter of any one of clauses 39-42, wherein:
   a first portion of the second conductive member is aligned with a third hole of the plurality of holes, wherein the first portion of the second conductive member and the conductive band form an electrode pair of a third shock wave emitter; and
   a second portion of the second conductive member is aligned with a fourth hole of the plurality of holes, wherein the second portion of the second conductive member and the conductive band form an electrode pair of a fourth shock wave emitter.
44. The catheter of any one of clauses 39-43, wherein the conductive member is aligned with a first three holes of the plurality of holes, thereby forming at least three shock wave emitters, and the second conductive member is aligned with a second three holes of the plurality of holes, thereby forming at least three different shock wave emitters.
45. The catheter of any one of clauses 37-44, wherein the second hole is positioned less than 180 degrees apart from the first hole around the circumference of the conductive band.
46. A catheter comprising:
   an outer sheath;
   an elongate member positioned at least partially within the outer sheath;
   a plurality of shock wave emitters mounted to a distal portion of the elongate member;
   an inner member positioned at least partially within the elongate member, wherein the inner member comprises a pre-formed distal portion configured such that in a deployed position distal of a distal end of the outer sheath, the pre-formed distal portion unfolds into an annular loop around at least a portion of a longitudinal axis of the catheter body,
   wherein the distal portion of the elongate member is configured to conform to the annular loop of the pre-formed distal portion.
47. The catheter of clause 46, wherein the elongate member is configured to translate relative to the inner member between an extended position and a retracted position, and the distal portion conforms to the annular loop of the pre-formed distal portion in the extended position.
48. The catheter of any one of clauses 46-47, wherein the elongate member is bonded to the pre-formed distal portion of the inner member, such that in the deployed configuration, the elongate member unfolds into the loop and the plurality of shock wave emitters are positioned along the loop.
49. The catheter of any one of clauses 46-48, wherein the pre-formed distal portion is configured such that in a retracted position within the outer sheath, the pre-formed distal portion folds into a folded configuration.
50. The catheter of any one of clauses 46-49, comprising an enclosure sealed to a distal end of the elongate member or the inner member.

1. (canceled)

2. A catheter for treating calcified tissue in a body with shock waves, the catheter comprising:
a first electrode pair and a second electrode pair, each of the first and second electrode pairs comprising a cathode and an anode, wherein the anode is formed from a more durable material than the cathode, wherein an anode of the first electrode pair is connected to a cathode of the second electrode pair;
a pair of conductors electrically connected to the first and second electrode pairs;
wherein the catheter is configured such that a voltage pulse applied across the pair of conductors causes current to arc from a cathode of the first electrode pair to the anode of the first electrode pair, thereby generating a first shock wave, and to arc from the cathode of the second electrode pair to an anode of the second electrode pair, thereby generating a second shock wave.

3. The catheter of claim 2, wherein the first electrode pair and the second electrode pair are circumferentially distributed about a longitudinal axis of the catheter and the first electrode pair and the second electrode pair are configured such that the first and second shock waves propagate radially away from the longitudinal axis.

4. The catheter of claim 2, wherein each anode of a respective pair of electrodes has a greater surface area than each cathode of the respective pair of electrodes.

5. The catheter of claim 2, wherein the anode of each of the first and second pairs of electrodes is formed by a conductive band.

6. The catheter of claim 5, wherein each conductive band comprises at least one elongate slot that extends at least partially around a circumference of the conductive band, and wherein each cathode of the first and second electrode pairs is aligned with the at least one elongate slot radially inward of the respective conductive band.

7. The catheter of claim 6, wherein the at least one elongate slot extends 60 degrees to 120 degrees around the circumference of the respective conductive band.

8. The catheter of claim 2, wherein the cathode of the first electrode pair is formed by an end of a first wire, and the cathode of the second electrode pair is formed by an end of a second wire.

9. The catheter of claim 8, wherein the second wire is connected to the anode of the first electrode pair.

10. The catheter of claim 8, wherein the second wire is soldered, welded, or clamped to the anode of the first electrode pair.

11. The catheter of claim 2, wherein the anode of the first electrode pair has at least one of a higher material hardness and a higher melting temperature than the cathode of the first electrode pair.

12. The catheter of claim 2, wherein the cathode of the first electrode pair is formed from copper.

13. The catheter of claim 2, wherein the anode of the first electrode pair is formed from molybdenum, tungsten, rhodium, rhenium, tantalum, niobium, or a combination thereof.

14. The catheter of claim 2, comprising an enclosure enclosing the first and second electrode pairs, the enclosure being fillable with a conductive fluid.

15. The catheter of claim 14, wherein at least a portion of the enclosure is configured to coil into an annular loop when the enclosure is filled with the conductive fluid.

16. The catheter of claim 14, wherein the enclosure is configured to occlude fluid flow through a cardiac valve when the enclosure is filled with the conductive fluid.

17. The catheter of claim 2, comprising an emitter support member that includes a pre-formed portion configured to coil into a loop, wherein the first and second electrode pairs are mounted to the pre-formed portion.

18. The catheter of claim 17, comprising an outer sheath, wherein the pre-formed portion is deployable from the outer sheath.

19. The catheter of claim 18, wherein the pre-formed portion is configured such that in a retracted position within the outer sheath of the catheter, the pre-formed portion folds into a folded configuration.

20. The catheter of claim 18, wherein, in a deployed position, the first electrode pair and the second electrode pair are positioned at a same longitudinal location with respect to the outer sheath.

21. The catheter of claim 20, wherein, in the deployed position, the first electrode pair is configured to generate shock waves biased in a particular direction and the first electrode pair is positioned relatively closer to a portion of an enclosure enclosing the first electrode pair that is opposite of the particular direction to prevent damage to the enclosure.
